(19) United States
(12) Patent Application Publication
(10) Pub. No.: US 2024/0139515 A1
(43) Pub. Date: May 2, 2024

(54) TRIALING USING PHYSIOLOGICAL SENSED SIGNALS FOR MEDICAL DEVICE IMPLANTATION

(71) Applicant: Medtronic, Inc., Minneapolis, MN (US)

(72) Inventors: Katelynn M. Johnson, New Brighton, MN (US); Simeng Zhang, Golden Valley, MN (US); Julia P. Slopsema, Minneapolis, MN (US); Lisa M. Jungbauer Nikolas, Lino Lakes, MN (US); Sarah J. Offutt, Golden Valley, MN (US)

(21) Appl. No.: 18/494,403

(22) Filed: Oct. 25, 2023

Related U.S. Application Data
(60) Provisional application No. 63/381,479, filed on Oct. 28, 2022.

(51) Int. Cl. A61N 1/36; A61B 5/00

(52) U.S. Cl. CPC A61N 1/36135; A61B 5/4848; A61N 1/36125; A61N 1/36167; A61N 1/36189

(57) ABSTRACT

An example method includes: delivering one or more electrical stimulation pulses to one or more nerves of a patient via one or more inserted electrodes on a lead or formed on an evaluation needle and prior to implantation of an implantable medical device, the one or more nerves being associated with stimulation therapy; sensing an evoked signal generated by one or more signal sources in response to delivery of the one or more electrical stimulation pulses; determining, based at least in part on the evoked signal, a therapy response group of a plurality of therapy response groups for the patient, wherein the therapy response group indicates whether implantation of the implantable medical device is recommended; and outputting information indicative of the determined therapy response group.

This application claims the benefit of U.S. Provisional Patent Application No. 63/381,479, filed 28 Oct. 2022, the entire contents of which is incorporated herein by reference.

### TECHNICAL FIELD

This disclosure generally relates to medical devices, and more specifically, electrical stimulation.

### BACKGROUND

Electrical stimulation devices, sometimes referred to as neurostimulators or neurostimulation devices, may be external to or implanted within a patient, and configured to deliver electrical stimulation therapy to various tissue sites to treat a variety of symptoms or conditions such as chronic pain, tremor, Parkinson's disease, epilepsy, or other neurological disorders, bladder dysfunction such as retention, overactive bladder, urgency, urgency frequency, urinary incontinence, bladder incontinence, bowel incontinence, fecal incontinence, sexual dysfunction, obesity, or gastroparesis. An electrical stimulation device may deliver electrical stimulation therapy via electrodes, e.g., carried by one or more leads, positioned proximate to target locations associated with the brain, the spinal cord, nerves of the pelvis and pelvic floor, tibial nerves, peripheral nerves, the gastrointestinal tract, or elsewhere within a patient. Stimulation proximate the spinal cord, proximate the sacral nerves, within the brain, and proximate peripheral nerves is often referred to as spinal cord stimulation (SCS), sacral neuromodulation (SNM), deep brain stimulation (DBS), and peripheral nerve stimulation (PNS), respectively.

A physician or clinician may select values for a number of programmable stimulation parameters in order to define the electrical stimulation therapy to be delivered by the implantable stimulator to a patient. For example, the physician or clinician may select one or more electrodes, polarities of selected electrodes, a voltage or current amplitude, a pulse width, a pulse frequency, a cycling, and a duration of stimulation as stimulation parameters. A set of therapy stimulation parameters, such as a set including electrode combination or configuration, electrode polarity, amplitude, pulse width, pulse shape, pulse frequency or pulse rate, or cycling may be referred to as a therapy program in the sense that they define the electrical stimulation therapy to be delivered to the patient.

### SUMMARY

This disclosure describes example techniques for determining whether a patient is a candidate for implantation of an implantable medical device based on sensed signals. The determination of the candidacy for implantation may be considered as a trialing period. In one or more examples, during a trialing period, a stimulator (e.g., external stimulator) may output a stimulation pulse that evokes a signal, referred to as an evoked signal. Processing circuitry may be configured to process the evoked signal, such as determine characteristics of the evoked signal. Based on the evoked signal, the processing circuitry may determine a therapy response group from a plurality of therapy response groups for the patient. As one example, one of the therapy response groups may be a group that indicates that implantation of an implantable medical device is recommended. One of the therapy response groups may be group that indicates that further trialing is recommended before determining whether to implant an implantable medical device.

In some examples, the groups may further define the type of implantable medical device. For instance, there may be three groups. A first group indicates that a non-rechargeable implantable medical device is recommended for the patient. A second group indicates that a rechargeable implantable medical device is recommended for the patient. A third group indicates that further trialing is recommended before determining whether to implant the implantable medical device.

In one or more examples, the evoked signal may be evoked coincident with the delivery of the stimulation pulse. Therefore, the processing circuitry may be configured to determine the therapy response group in a relatively short amount of time (e.g., less than one day, less than one hour, less than one minute, or less than one second). In the event that the processing circuitry determines the therapy response group as one of the groups for implanting the implantable medical device (e.g., rechargeable or non-rechargeable), a surgeon may complete the implantation procedures, rather than having the patient wait multiple days or weeks to determine whether implantation is recommended.

In one example, this disclosure describes a system that includes a device including processing circuitry configured to: deliver one or more electrical stimulation pulses to one or more nerves of a patient via one or more inserted electrodes on a lead or formed on an evaluation needle and prior to implantation of an implantable medical device, the one or more nerves being associated with stimulation therapy; sense an evoked signal generated by one or more signal sources in response to delivery of the one or more electrical stimulation pulses; determine, based at least in part on the evoked signal, a therapy response group of a plurality of therapy response groups for the patient, wherein the therapy response group indicates whether implantation of the implantable medical device is recommended; and output information indicative of the determined therapy response group.

In another example, this disclosure describes a method that includes: delivering one or more electrical stimulation pulses to one or more nerves of a patient via one or more inserted electrodes on a lead or formed on an evaluation needle and prior to implantation of an implantable medical device, the one or more nerves being associated with stimulation therapy; sensing an evoked signal generated by one or more signal sources in response to delivery of the one or more electrical stimulation pulses; determining, based at least in part on the evoked signal, a therapy response group of a plurality of therapy response groups for the patient, wherein the therapy response group indicates whether implantation of the implantable medical device is recommended; and outputting information indicative of the determined therapy response group.

In another example, this disclosure describes a computer readable medium that includes instructions that when executed cause one or more processors to: deliver one or more electrical stimulation pulses to one or more nerves of a patient via one or more inserted electrodes on a lead or formed on an evaluation needle and prior to implantation of an implantable medical device, the one or more nerves being associated with stimulation therapy; sense an evoked signal generated by one or more signal sources in response to the delivery of the one or more electrical stimulation pulses; determine, based at least in part on the evoked signal, a therapy response group of a plurality of therapy response groups for the patient, wherein the therapy response group indicates whether implantation of the implantable medical device is recommended; and output information indicative of the determined therapy response group.

The details of one or more examples are set forth in the accompanying drawings and the description below. Other features, objects, and advantages will be apparent from the description, drawings, and claims. The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating an example evaluation lead to determine if the patient is a candidate for implantation of an 1 MB, in accordance with one or more techniques of this disclosure.

FIG. 2 is a conceptual diagram illustrating an example system that includes an implantable medical device (IMD) in the form of a neurostimulation device configured to deliver sacral neuromodulation (SNM), an external programmer, and one or more sensing devices in accordance with one or more techniques of this disclosure.

FIG. 3 is a block diagram illustrating an example of an 1 MB in the form of a neurostimulation device, in accordance with one or more techniques of this disclosure.

FIG. 4A is a flow diagram illustrating an example method of determining a therapy response group for a patient, in accordance with one or more techniques of this disclosure.

FIG. 4B is a flow diagram illustrating another example method of determining a therapy response group for a patient, in accordance with one or more techniques of this disclosure.

FIG. 5 is a plot of an example evoked signal, in accordance with one or more techniques of this disclosure.

FIG. 6 is a plot of another example evoked signal, in accordance with one or more techniques of this disclosure.

FIG. 7 is a plot of another example evoked signal, in accordance with one or more techniques of this disclosure.

FIG. 8 is a plot of another example evoked signal, in accordance with one or more techniques of this disclosure.

FIG. 9 is a plot of another example composite evoked signal, in accordance with one or more techniques of this disclosure.

FIG. 10 is a flow diagram illustrating an example method of determining a therapy response group for a patient indicating a therapy device type, in accordance with one or more techniques of this disclosure.

FIG. 11 is a flow diagram illustrating another example method of determining a therapy response group for a patient indicating a therapy device type, in accordance with one or more techniques of this disclosure.

FIG. 12 is a flow diagram illustrating an example method of determining a therapy response group for a patient indicating a nerve to use for pelvic disorder therapy, in accordance with one or more techniques of this disclosure.

### DETAILED DESCRIPTION

Electrical stimulation therapy, e.g., sacral nerve stimulation, tibial nerve stimulation, and/or other types of invasive or noninvasive neuromodulation, may provide neurological disorder therapy (e.g., pelvic disorder therapy, excretory system dysfunction therapy), pain relief and/or other therapeutic benefits. Before implantation of an implantable medical device (IMD) for therapy, a patient undergoes a trialing period to determine whether implantation of the IMD is recommended. During this trialing period, an external neurostimulator (ENS) stimulates nerves of the patient to see how the patient reacts to stimulation. For instance, the patient may maintain a diary or other record indicating the number of incontinence events, number of times the patient relieved himself/herself, etc. when stimulation is being delivered. This trialing period helps determine if an IMD would provide effective therapy. However, methods for some stimulation trialing periods may take weeks to determine whether the patient is a candidate for an implantable medical device.

In accordance with one or more techniques of this disclosure, example stimulation systems and example techniques may utilize evoked signals (e.g., stimulation-evoked signals) for determining a therapy response group from a plurality of therapy response groups for patients (e.g., classifying patients into therapy response groups) substantially coincident with the delivery of one or more electrical stimulation pulses in an outpatient procedure. For example, electrical stimulation may evoke a response (e.g., a signal) such as a neural response of one or more nerves and/or contractions of one or more muscles. For example, stimulation of sacral nerves through electrical leads near sacral nerves via sacral neuromodulation may evoke a neural response in adjacent nerves, muscle contractions within the pelvic floor, and distal contractions in the foot. The neural response in nerves and/or activation/contraction of muscles evoked by electrical stimulation may be captured (e.g., or detected, sensed, measured, and the like) as one or more evoked signals. In some examples, captured evoked signals may be a composite of multiple signals evoked by multiple signal sources (e.g., nerves and/or muscles) in response to delivery of electrical stimulation therapy. However, the captured evoked signals should not be considered limited to composite of multiple signals.

An evoked signal during trialing may include one or more features that may indicate one or more aspects of electrical stimulation therapy delivery, such as whether the patient is a candidate for an implantable medical device for delivering neurological disorder therapy. The systems and techniques of this disclosure may indicate whether the patient is a candidate for an implantable medical device for delivering neurological disorder therapy, as well as what type of device and nerve are most advantageous for delivering neurological disorder therapy. Evoked signals that satisfy one or more criteria in response to electrical stimulation from an evaluation needle/lead may indicate that the patient can be classified into one or more therapy response groups, e.g., groups indicative of a medical device type effective for therapy and/or an effective therapy program for delivering neurological disorder therapy to the patient. That is, the evoked signals that satisfy one or more criteria in response to electrical stimulation from an evaluation needle/lead may indicate that the patient can be implanted with an implantable medical device. In this way, physicians may more quickly determine effective treatment programs for patients, using fewer hospital resources, and through less patient discomfort and burden. Moreover, if determined that the patient is to be implanted with the implantable medical device, a surgeon can complete the implantation procedure at the time the evoked signals are processed, allowing to delivery of therapy at an earlier time compared to requiring multiple days or weeks for trialing. The surgeon may guide a permanent lead of the implantable medical device to the location where evoked signals satisfied the one or more criteria and make minor adjustments until evoked signals from the permanent lead substantially match evoked signals sensed from the evaluation lead/needle. Throughout the disclosure, reference to “neurological disorder therapy” should not necessarily be interpreted to mean that the disorder is “neurogenic,” that is, that the disorder originates in nerves or nerve tissue. Instead “neurological disorder therapy” is used herein to refer to therapy provided by way of stimulation to nerves or nerve tissue wherein the underlying disorder or symptoms being treated may or may not originate in nerves or nerve tissue. Neurological disorders may include pain disorders.

In some examples, the system may be configured to determine the one or more criteria for evoked signals based on the features and/or a collection of features captured from a collection of patients. For example, machine learning may be used on a collection of features from patient signals paired with stimulation outcome measures to build a classification algorithm that can predict patient therapy response outcome and therapy efficacy. In some examples, the predicted therapy efficacy may then be used to make therapy decisions, e.g., implant the medical device or not implant the medical device, choose battery types for an implantable medical device, choose which nerves to stimulate for neurological disorder therapy, tune stimulation parameters, and the like.

Systems and methods for determining a therapy response group (e.g., classifying a patient into a therapy response group) based on an evoked signal are described herein. The system may include a stimulator device that interacts with an external programmer. Various examples are discussed relative to one or more stimulation devices. It is recognized that the stimulation devices may include features and functionality in addition to electrical stimulation. Many of these additional features are expressly discussed herein. A few example features include, but are not limited to, different types of sensing capabilities and different types of wireless communication capabilities. For ease of discussion, the present disclosure does not expressly recite every conceivable combination of the additional features, such as by repeating every feature each time different examples and uses of the stimulation devices are discussed.

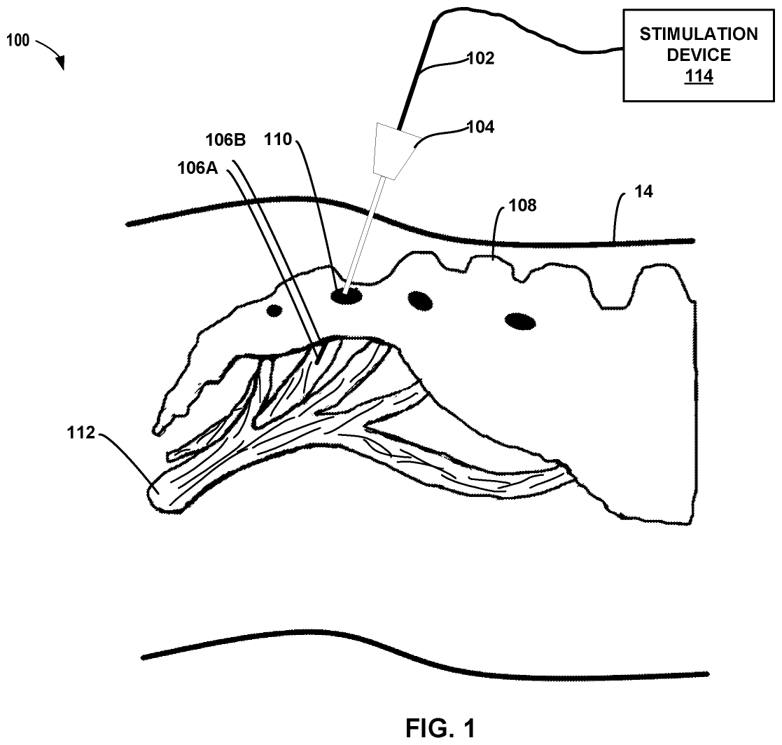

FIG. 1 is a conceptual diagram illustrating an example evaluation lead system 100, in accordance with one or more techniques of this disclosure. In the example of FIG. 1, evaluation lead system 100 includes lead 102, introducer 104, and electrodes 106A and 106B. As described below, lead 102 is one example, and rather than or in addition to lead 102, system 100 may include an evaluation needle configured to perform the example operations described for lead 102. Accordingly, this disclosure may refer to reference numeral 102 as lead 102 and/or evaluation needle 102.

Lead 102 may be attached to a stimulation device 114, and may be an external neurostimulator (ENS). In some examples, it may be possible that stimulation device 114 is an implantable medical device (IMD) that is used as an ENS prior to implantation. Stimulation device 114 may be configured to deliver one or more electrical stimulation pulses to one or more nerves 112 of patient 14 via electrode 106A and prior to implantation of an implantable medical device. The one or more nerves 112 are associated with pelvic disorder therapy. In some examples, evaluation lead system 100 may include an evaluation needle composed of a conductive material, wherein the conductive material is exposed near a distal end and is configured to deliver the one or more electrical stimulation pulses and sense evoked signals, as described below. In some examples, the evaluation needle may be used in lieu of or in addition to lead 102, introducer 104, and electrode 106A and 106B to perform the capabilities attributed to lead 102 as described herein. Lead 102 and/or the evaluation needle may be inserted through foramen 110 in spine 108 of patient 14 to locate electrode 106A near or adjacent to nerves 112 of patient 14. Throughout the disclosure, reference may be made to an evaluation needle including an “electrode” for sensing and stimulation. In some examples, an evaluation needle may include an exposed conductive surface that functions as an electrode to deliver one or more electrical stimulation pulses and sense one or more evoked signals.

In the illustrated example, lead 102 includes electrode 106A used for stimulation and electrode 106B used for sensing. However, the example techniques are not so limited. In some examples, there may be only one electrode 106 used for stimulation and sensing. As another example, lead 102 includes electrode 106A, and another implanted lead (not shown) may include electrode 106B for sensing. As another example, electrode 106B may be a patch electrode or some other electrode that is coupled to the skin of the patient. The location of electrodes 106A and 106B being on lead 102 is one example, and should not be considered as limiting.

Although not illustrated, in some examples, lead 102 (and/or evaluation needle 102) may include one or more reference electrodes (e.g., ground electrode). The one or more reference electrodes may provide a return path for the stimulation pulses from electrode 106A, or may provide a reference for signals sensed by electrode 106B. In some examples, in addition to or instead of reference electrodes on lead 102 or evaluation needle 102, stimulation device 114 may include the reference electrodes. Other examples of reference electrodes are possible such as reference electrodes attached to the skin of patient 14.

Evaluation lead system 100 may be used to determine if patient 14 is a candidate for implantation of an IMD to provide long-term pelvic disorder therapy (e.g., bladder and/or bowel dysfunction therapy). Prior to implantation of the IMD for long term therapy, a physician determines whether implantation of the IMD will provide effective therapy for patient 14. Evaluation lead system 100 may determine if patient 14 is a candidate for implantation of an IMD for long-term therapy.

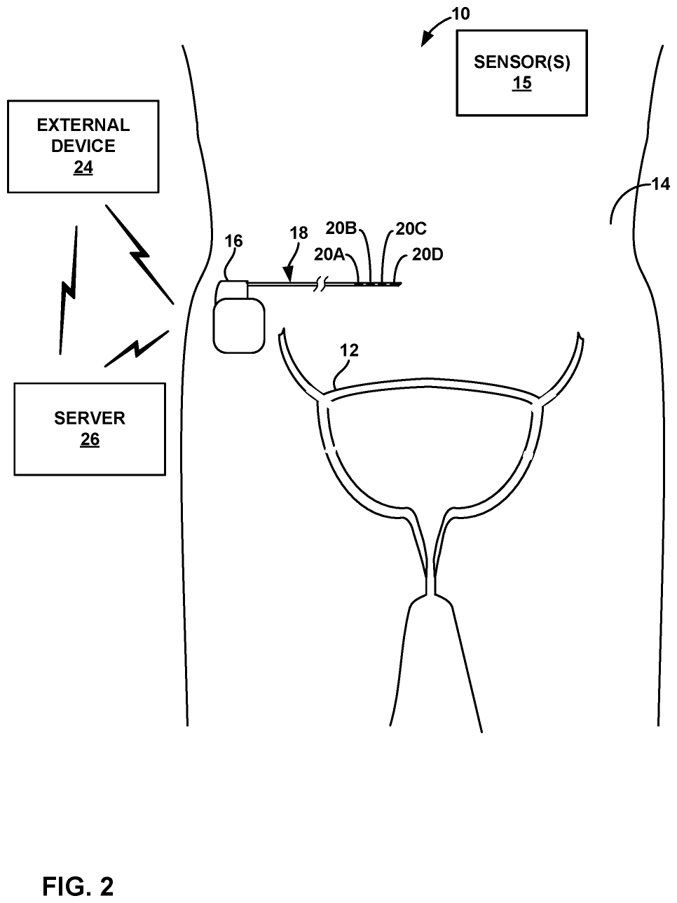

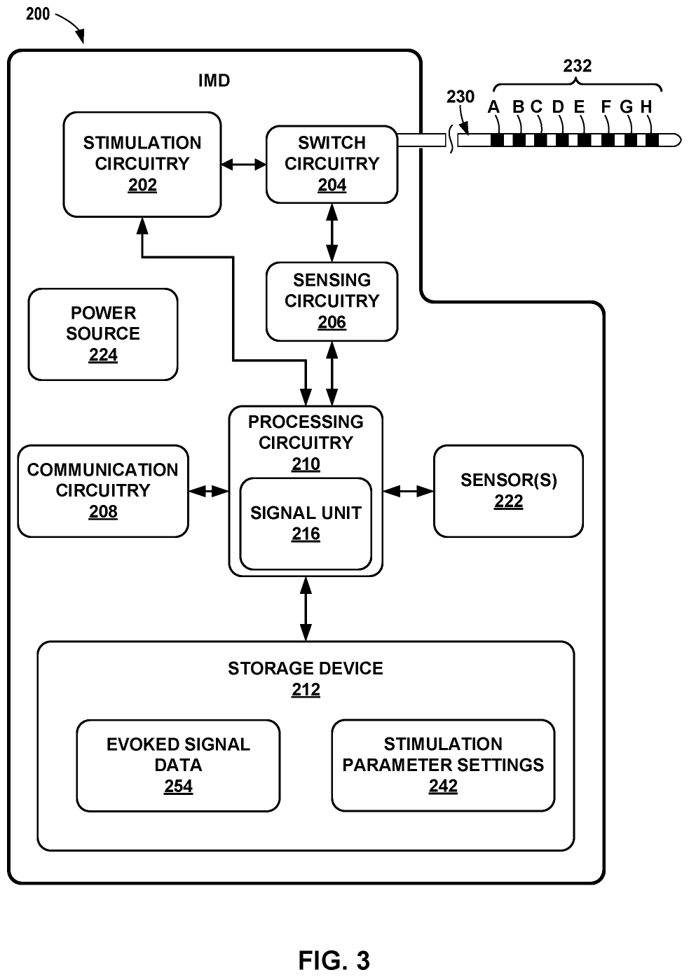

Stimulation device 114 of evaluation lead system 100 may include processing circuitry configured to deliver one or more electrical stimulation pulses to nerves 112 of patient 14 via electrode 106A. When reference is made to “processing circuitry” performing certain functions, it may be understood that processing circuitry performs these functions in tandem with other relevant circuitry (e.g., stimulation circuitry 202 as described in FIG. 3). Furthermore, although described as processing circuitry of stimulation device 114, in some examples the processing circuitry may be part of a different computing device in communication with stimulation device 114 (e.g., an external device and/or server as described below), or a combination of processing circuitry of multiple devices. The one or more nerves may be one or more nerve 112 as described below reference to FIGS. 2 and 3 (e.g., sacral nerves, sciatic nerves, tibial nerves, etc.). The processing circuitry may also be configured to sense, via electrode 106B, one or more evoked signals generated by one or more signal sources (nerves or muscles) in response to delivery of the one or more electrical stimulation pulses.

Based at least in part on the one or more evoked signals, the processing circuitry of stimulation device 114 may classify patient 14 into a therapy response group that indicates at least one of a therapy device type and a therapy program for delivering pelvic disorder therapy to patient 14. A therapy device type may include one or more different device types, for example an implantable medical device, a rechargeable implantable medical device, a non-rechargeable implantable medical device. A therapy program may include one or more different treatment programs, for example a trialing program, and/or no therapy.

In other words, the processing circuitry may determine, based at least in part on the evoked signal, a therapy response group of a plurality of therapy response groups for the patient. The therapy response group may indicate whether implantation of the implantable medical device is recommended. The processing circuitry may output information indicative of the determined therapy response group. As one example, there may be two therapy response groups: a first therapy response group indicates that implantation of an implantable medical device is recommended, and a second therapy response group indicates that further trialing is needed before determining that implantation of an implantable medical device is recommended. As another example, there may be three therapy response groups: a first therapy response group indicates that implantation of a rechargeable implantable medical device is recommended, a second therapy response group that indicates that implantation of a non-rechargeable implantable medical device is recommended, and a third therapy response group indicates that further trialing is needed before determining that implantation of an implantable medical device is recommended.

Accordingly, the processing circuitry may determine the therapy response group for the patient, which indicates whether implantation of the implantable medical device is recommended. That is, based on the evoked signal, the processing circuitry may classify patient 14 into a first therapy response group (implantation of an implantable medical device is recommended) or into a second therapy response group (further trialing is needed). As another example, based on the evoked signal, the processing circuitry may classify patient 14 into a first therapy response group (implantation of a rechargeable implantable medical device is recommended), into a second therapy response group (implantation of a non-rechargeable implantable medical device is recommended), or into a second therapy response group (further trialing is needed).

The processing circuitry may classify patient 14 into a therapy response group (e.g., determine the therapy response group) substantially coincident with the delivery of the one or more electrical stimulation pulses. For example, the processing circuitry may classify patient 14 into a therapy response group in a time period after delivery of the one or more electrical stimulation pulses. In some examples, the time period may be less than one day, less than one hour, less than one minute, and/or less than one second. By classifying patient 14 into a therapy response group within such a short time period, system 100 may allow a physician to determine if patient 14 is a candidate for therapy by an IMD in a single patient procedure, rather than requiring patient 14 to wear a trialing lead for weeks. In some examples, if determined that implantation is appropriate, the physician may perform the implantation while patient 14 is at the physician's office or hospital.

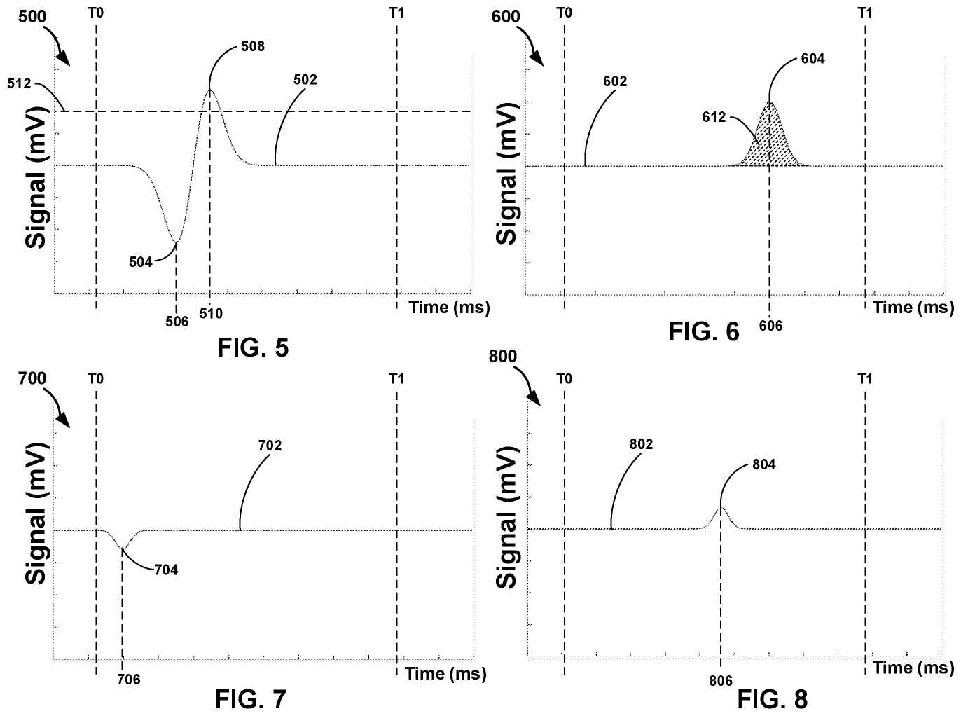

In order to classify patient 14 into a therapy response group (e.g., determine the therapy response group for patient 14) substantially coincident with the delivery of the one or more electrical stimulation pulses, the processing circuitry may be configured to determine whether the evoked signal satisfies one or more criteria. For example, the evoked signal (e.g., as received from sensing electrode 106B) may be sensed or recorded by stimulation device 114 as signal data, which may include a time-varying signal indicative of a response or responses of one or more signal sources (e.g., nerves and/or muscles) to the electrical stimulation, discussed in more detail below with reference to FIGS. 5-9. In some examples, in response to determining that the evoked signal satisfies one or more criteria, the processing circuitry may classify patient 14 into a therapy response group that indicates an IMD as the therapy device type for delivering pelvic disorder therapy to patient 14 (e.g., determine that implantation of an IMD is recommended for patient 14). For example, the processing circuitry may classify patient 14 into a therapy response group that indicates that pelvic disorder therapy provided by an implanted IMD (e.g., IMD 16) would be effective for alleviating pelvic disorder symptoms of patient 14.

In some examples, in response to determining that the evoked signal does not satisfy one or more criteria, the processing circuitry may classify patient 14 into a therapy response group that indicates the patient is a candidate for a longer term evaluation of the candidacy of patient 14 for pelvic disorder therapy (e.g., determine that further trialing is needed). Longer term evaluation of the candidacy of the patient may include multiple-day determinations of the efficacy of pelvic disorder therapy, e.g., by way of a trialing device that is not implanted. For example, the processing circuitry may classify patient 14 into a therapy response group that indicates that processing circuitry is unable to determine whether full implantation of an IMD for pelvic disorder therapy would be effective for patient 14.

In some examples, in response to determining that the evoked signal does not satisfy one or more criteria, the processing circuitry may classify patient 14 into a therapy response group that indicates the patient is not a candidate for pelvic disorder therapy. For example, processing circuitry may classify patient 14 into a therapy response group that indicates patient 14 would not benefit significantly from therapy provided by an implanted IMD.

In some examples, in response to determining that the evoked signal satisfies one or more criteria, the processing circuitry may classify patient 14 into a therapy response group that indicates a nerve of the patient to provide pelvic disorder therapy. For example, the processing circuitry may classify the patient into a therapy response group that indicates that stimulation of a sacral nerve would be an effective stimulation nerve for pelvic disorder therapy. In some examples, the processing circuitry may classify the patient into a therapy response group that indicates that stimulation of a tibial nerve would be an effective stimulation nerve for pelvic disorder therapy. In some examples, the processing circuitry may classify the patient into a therapy response group that indicates that stimulation of muscle tissue would be effective for pelvic disorder therapy. In some examples, the processing circuitry may classify the patient into a therapy response group that indicates that stimulation of a certain part of a nerve would be effective for pelvic disorder therapy. In some examples, the processing circuitry may classify the patient into a therapy response group that indicates a particular branch of a sacral nerve would be an effective stimulation nerve for pelvic disorder therapy. In some examples, the processing circuitry may classify the patient into a therapy response group that indicates nerves on a left side or a right side of the patient as effective stimulation nerves for pelvic disorder therapy.

In some examples, the processing circuitry may indicate that it cannot classify patient 14 into a therapy response group based on the evoked signal received from electrode 106B with the current stimulation location of electrode 106A. The processing circuitry may indicate that a location of lead 102 and electrode 106A should change in order to better classify patient 14 into a therapy response group. For example, the processing circuitry may deliver a first set of one or more electrical stimulation pulses to nerves 112 of patient 14 via electrode 106A located at a first location within patient 14. The processing circuitry may then sense, via electrode 106B, a first evoked signal generated by nerves 112 with electrode 106A at the first location in response to the first set of one or more electrical stimulation pulses. The processing circuitry may determine that the first evoked signal does not satisfy one or more criteria. In some examples, there may be only one electrode 106 used for stimulation and sensing. In some examples, the single electrode for stimulation may be an exposed tip of an evaluation needle.

The physician may then move lead 102. The processing circuitry may then deliver a second set of one or more electrical stimulation pulses to nerves 112 or other nerves of patient 14 at a second location within patient 14. The processing circuitry may then determine if a second evoked signal, sensed via electrode 106B, from delivery of the second set of one or more electrical stimulation pulses satisfies one or more criteria. The processing circuitry may classify patient 14 into a therapy response group based at least in part on whether the second set of one or more electrical stimulation pulses satisfies the one or more criteria. In some examples, this procedure may repeat until processing circuitry is able to classify the patient into a therapy response group. In between delivery of sets of electrical stimulation pulses, a physician may relocate electrode 106A and/or lead 102.

In some examples, after the processing circuitry classifies patient 14 into a therapy response group that indicates that patient 14 is a candidate for pelvic disorder therapy from an IMD, the physician may proceed to implant a permanent lead and an IMD. The physician may guide the permanent lead of the implantable medical device to the location where evoked signals satisfied the one or more criteria and adjust placement of the permanent lead until evoked signals from the permanent lead substantially match evoked signals sensed from the evaluation lead/needle.

In some examples, the processing circuitry may classify patient 14 into a therapy response group based on the evoked signal received from electrode 106B with a first stimulation location of electrode 106A, e.g., a sacral nerve branch on the left side of patient 14. The processing circuitry may save the data involved in classifying the patient to a database in memory. A physician may relocate electrode 106A, electrode 106B and/or lead 102 to a second stimulation location, e.g., to a sacral nerve branch on the right side of patient 14. Processing circuitry may then deliver a set of one or more electrical stimulation pulses to the nerves at the second stimulation location. The processing circuitry may classify patient 14 into a therapy response group based on the evoked signal received from electrode 106B with the second stimulation location of electrode 106A. The processing circuitry may be configured to compare the first classification of patient 14 in memory at the first location to the second classification of patient 14 at the second location and determine which location is a preferred location for pelvic disorder therapy for patient 14.

In some examples, the evoked signal data (e.g., stimulation-evoked signal data) may include an averaged signal and/or one or more signal features determined via processing of the signal, e.g., peak/valley detection, peak/valley amplitude, width, and/or area, frequency analysis, digital signal processing, signal latency, and the like. In some examples, the one or more signal features may include one or more of a signal peak, a signal peak amplitude, a number of signal peaks, an area under one or more signal peaks, a signal peak width, a time between signal peaks, a ratio of signal peak amplitudes, a ratio of signal peak widths, a ratio of areas under signal peaks, a latency of a signal peak, a signal valley, a signal valley amplitude, a number of signal valleys, an area above one or more signal valleys, a signal valley width, a time between signal valleys, a ratio of signal valley amplitudes, a ratio of signal valley widths, a ratio of areas above signal valleys, a valley latency, a root-mean-square signal value, a signal skew, a signal kurtosis, a signal frequency, a signal spectral content, a Hjorth feature, a signal amplitude growth curve threshold, a signal amplitude growth curve inflection point amplitude, a signal amplitude growth curve inflection point latency, a signal amplitude growth curve saturation point, a signal strength duration curve chronaxie, a signal strength duration curve rheobase, or another signal strength duration curve feature, a signal maximum rate of change feature (e.g., maximum of the derivative of the signal), or a signal minimum rate of change feature (e.g., the minimum of the derivative of the signal), or any other suitable signal feature. Criteria for the evoked signal data based on these signal features may be stored in memory of stimulation device 114 or another device in communication with stimulation device 114. In some examples, criteria for the evoked signal data may be adaptive, and change based on a database of received and/or stored evoked signal data. For example, a computing device in communication with a plurality of stimulation devices may be configured to collect and store a plurality of evoked signal data from a plurality of patients, and correlate the plurality of evoked signal data with therapy response groups and/or therapy outcomes for the respective patients to whom the evoked signal data correlates.

In some examples, the computing device may determine the one or more criteria for the evoked signal based on a trained machine learning model. For example, the computing device may collect the plurality of evoked signal data from a plurality of patients that have found therapy from particular therapy programs as being effective. For example, the computing device may collect the plurality of evoked signal data from a plurality of patients that have found therapy from an implanted IMD as being effective. In some examples, the computing device may collect the plurality of evoked signal data from a plurality of patients that have found therapy from an implanted IMD with a rechargeable battery as being effective. In some examples, the computing device may collect the plurality of evoked signal data from a plurality of patients that have found therapy from an implanted IMD with a non-rechargeable battery as being effective. In some examples, the computing device may collect the plurality of evoked signal data from a plurality of patients that have found therapy from an implanted IMD as being effective when stimulating a particular nerve. The computing device may train the machine learning model using evoked signal data as input for patients that found therapy from an IMD as being effective. That is, evoked signals from patients that have found therapy from an IMD as being effective may be “ground truths” for training a machine learning model (e.g., neural network, convolutional neural network, etc.) The computing device may update weights of nodes within the machine learning model based on the ground truths to generate a trained machine learning model. The processing circuitry may utilize the trained machine learning model, and input sensed evoked signals. The output from the trained machine learning model may be a determination a therapy response group for whether implantation of an IMD, and possibly a particular IMD type, is recommended for a patient. That is, the trained machine model may classify patient 14 into a therapy response group of a plurality of therapy response groups.

In some examples, the computing device may collect the plurality of evoked signal data from a plurality of patients that have found therapy from an IMD (whether implanted or not) as being ineffective. The computing device may train the machine learning model using evoked signal data as input for patients that found therapy from an IMD as being ineffective. In this way, the machine learning model may be trained to determine whether therapy from an IMD will be effective or not for a patient (e.g., classify the patient into a therapy response group of a plurality of therapy response groups). In some examples, if the machine learning model is unable to determine whether therapy from an IMD will be effective or not for a patient to a degree of certainty, machine learning model may classify the patient into a therapy response group that indicates that the patient is a candidate for longer term evaluation of whether to receive therapy with the IMD.

The computing device may be configured to periodically retrain the machine learning model over time (e.g., every six months) or when prompted by a user of the computing device. The computing device may retrain the machine learning model using evoked signal data collected since the previous training of the machine learning model. In some examples, the machine learning model may be stored in memory of stimulation device 114.

The example of FIG. 1 is described with respect to nerve 112, which may be a sacral nerve. However, the example techniques are not so limited. In some examples, stimulation device 114 may be used to stimulate a tibial nerve to evoke the evoked signal, and determine whether implantation of an IMD for stimulating the tibial nerve is appropriate. For stimulating the tibial nerve, the physician may insert lead 102 (or an evaluation needle) near the ankle of patient 14. It may be understood that references to lead 102 include examples where lead 102 is an evaluation needle. If determined that tibial nerve stimulation with an IMD would be effective, the implantation of the IMD may be near the ankle of patient 14 with a leadless or leaded device.

As described above, the processing circuitry may use sensed evoked signals to determine a therapy response group for patient 14 (e.g., classify patient 14 into a therapy response group indicating whether implantation of an IMD is recommended). The following describes examples of evoked signals.

The processing circuitry may be configured to sense, via electrode 106B, an evoked signal from nerve tissue of patient 14. In some examples, electrode 106B may be configured to sense and capture a composite stimulation-evoked signal comprising a composite of signals generated by two or more signal sources in response to the one or more electrical stimulation pulses. The example techniques do not require composite stimulation-evoked signals. In this disclosure, the term evoked signal includes a stimulation-evoked signal and a composite stimulation-evoked signal, where a composite stimulation-evoked signal is a combination of two or more stimulation-evoked signals.

In some examples, one or more signal sources, such as one or more nerves, one or more muscles, or at least one muscle and at least one nerve, may respond to the electrical stimulation, e.g., via a neural response, a muscle contraction and/or activation, or any other response. In some examples, the response of the one or more sources may be electrical, e.g., an ECAP, an EMG or surface EMG, and the like. In some examples, the response may be mechanical and converted to an electrical signal by a sensor or detector, e.g., by a piezoresistive sensor or other sensor configured to measure muscle contraction and mechanomyography (MMG) and the like. In some examples, nerves may include one or more of the sacral nerves and their branches, e.g., dorsal and ventral rami of sacral nerves, pudendal nerves, sciatic nerves, saphenous nerves, nerves in the sacral plexus, pelvic nerves, pelvic plexus nerves, pelvic splanchnic nerves, tibial nerves, inferior hypogastic plexus nerves, lumbosacral trunk nerves, e.g., where the lumbosacral trunk joins sacral nerves, any sympathetic nerve fibers in the sympathetic chain of any of the above nerves or other nerves. In some examples, one or more muscles may include an external anal sphincter muscle, coccygeus muscle, levator ani muscle group, bulbocavernosus and/or bulbospongiosus muscle, gluteal muscles, e.g., gluteal maximus, gluteal medius, and gluteal minimus, perineal muscles, ischiocavernosus muscles, puborectalis muscles, piriformis muscles, or any other muscles.

In some examples, an evoked signal sensed by one or more sensors and/or electrodes (e.g., like electrode 106B) may be a combination of any and/or all of the various signal sources. For example, an electrical stimulation pulse may cause a nerve and/or muscle proximate to the stimulation pulse to generate a response and other nerves or muscles, not necessarily proximate to the stimulation pulse, may also generate responses. The composite stimulation-evoked signal may be a composite of signals from any of the multiple signal sources.

Electrode 106B may receive and/or sense evoked signals from the one or more signal sources. In some examples, electrode 106B may receive and/or sense evoked signals from multiple signal sources evoked by the same electrical stimulation pulse. In some examples, the signals from multiple signal sources may “arrive” at electrode 106B at slightly different times. For example, a first evoked signal may arrive at electrode 106B four ms after the electrical stimulation pulse, and a second evoked signal may arrive at electrode 106B seven ms after the electrical stimulation pulse. In some examples, the received signals may be a composite signal where electrode 106B may receive and/or sense the signals from two or more signal sources concurrently over a period of time as a single composite stimulation-evoked signal. For example, two or more signals may “arrive” at electrode 106B at the same time and may add together forming the composite signal that is sensed. For example, the two or more signals may be electric signals which may add incoherently, coherently, constructively, destructively, and the like, to form the electric signal that is sensed. In other examples, the two or more signals may be individually sensed and then added and/or combined to form the composite stimulation-evoked signal. Again, in some examples, composite stimulation-evoked signals may not be needed, and electrode 106B may sense one evoked signal from one source.

In some examples, the two or more signal sources may be located relatively far from electrode 106B and/or each other, e.g., at least 5 millimeters (mm) from electrode 106B and/or each other, at least 10 mm from electrode 106B and/or each other, at least 100 mm from electrode 106B and/or each other, at least 200 mm from electrode 106B and/or each other, at least 1 meter from electrode 106B and/or each other. For example, two or more signal sources may include a tibial nerve and a sacral nerve responding to sacral nerve stimulation.

In some examples, the evoked signal may have a relative long duration, e.g., more than 5 milliseconds (ms), more than 10 ms, more than 20 ms, etc. For example, because the composite stimulation-evoked signal may originate from multiple signal sources at multiple distances from electrode 106B, and because different signal sources may have different response times, the signals from the signal sources may arrive at, and be captured by electrode 106B at different times. In some examples, electrode 106B may sense signals from signal sources after delivery of every electrical stimulation pulse, or electrode 106B may sense signals from signal sources after an amount of time after delivery of electrical stimulation pulses.

In some examples, the evoked signal may comprise signals of different types from different signal sources. For example, the composite stimulation-evoked signal may comprise an ECAP signal generated relatively quickly after delivery of electrical stimulation pulses, e.g., within 2 ms, and an EMG signal generated relatively slowly after delivery of electrical stimulation pulses, e.g., after 1 ms. In some examples, the composite stimulation-evoked signal may comprise signals from multiple signal sources that do not overlap in time. For example, the composite stimulation-evoked signal may comprise an ECAP signal from a signal source relatively close to the sensor and/or electrode followed by an EMG signal or another ECAP signal from a different signal source that may be relatively far from electrode 106B, e.g., such that the ECAP from the close signal source is no longer present while the EMG signal and/or ECAP from the more distant signal source are received by electrode 106B.

FIG. 2 is a conceptual diagram illustrating an example system 10 that includes an implantable medical device (IMD 16) in the form of a neurostimulation device configured to deliver sacral neuromodulation (SNM), an external programmer, and one or more sensing devices in accordance with one or more techniques of this disclosure. Although described for SNM, the example techniques are also applicable for other nerves associated with pelvic disorder, such as the tibial nerve, and for providing tibial neuromodulation (TNM). For TNM, IMD 16 may not be implanted as illustrated in FIG. 2, and may instead be implanted near the ankle of patient 14. For ease, the examples are described with respect to SNM with the understanding that the example techniques may be extended to other nerves, with other locations of IMD 16, and other classes of IMD 16 (e.g., leadless IMD).

In some examples, system 10 may determine one or more stimulation setting(s) and manage delivery of neurostimulation to patient 14, e.g., to manage pelvic disorders (e.g., bladder and/or bowel dysfunction therapy), such as retention, overactive bladder, urgency, urgency frequency, urinary incontinence, bladder incontinence, stress incontinence, nocturia, bowel incontinence, fecal incontinence, intractable constipation, irritable bowel syndrome, inflammatory bowel disease, neurogenic bowel and bladder (tremor, Parkinson's disease, epilepsy, multiple sclerosis, stroke, spinal cord injury, neuropathy), sexual dysfunction, obesity, gastroparesis, pelvic pain, chronic pain, and interstitial cystitis. As shown in the example of FIG. 2, therapy system 10 includes an implantable medical device (IMD) 16 (e.g., an example medical device), which is coupled to lead 18. System 10 also includes an external device 24, which is configured to communicate with IMD 16 via wireless communication. System 10 also includes server 26 which may be one or more servers in a cloud computing environment. Server 26 may be configured to communicate with external device 24 and/or IMD 16 via wireless communication through a network access point (not shown in FIG. 1) and may be collocated with external device 24 or may be located elsewhere, such as in a cloud computing data center.

IMD 16 generally operates as a therapy device that delivers neurostimulation (e.g., electrical stimulation in the example of FIG. 1) to, for example, a target tissue site proximate a spinal nerve, a sacral nerve, a sacral nerve branch, a pudendal nerve, dorsal genital nerve, a tibial nerve, a saphenous nerve, an inferior rectal nerve, a perineal nerve, or other pelvic nerves, branches of any of the aforementioned nerves, roots of any of the aforementioned nerves, ganglia of any of the aforementioned nerves, or plexus of any of the aforementioned nerves. IMD 16 provides electrical stimulation to patient 14 by generating and delivering a programmable electrical stimulation pulse (e.g., in the form of electrical pulses or an electrical signal) to a target a therapy site near lead 18 and, more particularly, near electrodes 20A-20D (collectively referred to as “electrodes 20”) disposed proximate to a distal end of lead 18.

In the example of FIG. 2, IMD 16 is implanted for stimulating the sacral nerve with lead 18. However, the location of IMD 16 may be different based on which nerve is being stimulated. For instance, IMD 16 may be implanted near the ankle for tibial stimulation, and IMD 16 may not include leads with electrodes. Instead, the electrodes used for stimulation may be formed on IMD 16 for tibial nerve stimulation.

Once it is determined that a patient is a candidate for implantation of IMD 16 (e.g., based on example techniques described above with respect to FIG. 1), IMD 16 may be surgically implanted in patient 14 at any suitable location within patient 14, such as near the pelvis or ankle (e.g., for tibial stimulation). In some examples, IMD 16 may be implanted in a subcutaneous location in the side of the lower abdomen or the side of the lower back or upper buttocks. IMD 16 has a biocompatible housing, which may be formed from titanium, stainless steel, a liquid crystal polymer, or the like. The proximal ends of lead 18 are both electrically and mechanically coupled to IMD 16 either directly or indirectly, e.g., via respective lead extensions. Electrical conductors disposed within the lead bodies of lead 18 electrically connect sense electrodes (e.g., electrodes 20A and 20B) and stimulation electrodes, such as electrodes 20C and 20D, to sensing circuitry and a stimulation delivery circuitry (e.g., a stimulation generator) within IMD 16.

The example of lead 18 and electrodes 20A-20D is provided as one example. In some examples, IMD 16 may be coupled to additional leads, such as leads having electrodes positioned for sensing an impedance of bladder 12, which may increase as the volume of urine within bladder 12 increases. In some examples, system 10 may include electrodes, a strain gauge, one or more accelerometers, ultrasound sensors, optical sensors, or any other sensor. In some examples, the sensors may be configured to gather information relating to the patient, such as detect contractions of bladder 12, pressure or volume of bladder 12, or any other indication of the fill cycle of bladder 12 and/or possible bladder dysfunctional states. In some examples, System 10 may use sensors for sensing information relating to the patient, such as bladder volume. System 10 may use the sensor data for determining stimulation program settings for a given patient. IMD 16 may communicate sensed data to server 26. In some examples, IMD 16 may communicate the sensor data through external device 24. In other examples, IMD 16 may communicate the sensor data to server 26 without communicating the sensor data through external device 24.

In some examples, external device 24 may collect user input identifying a voiding event, perceived level of fullness, or any other indication of an event associated with the patient. The user input may be in the form of a voiding journal analyzed by external device 24, IMD 16 or server 26, or individual user inputs associated with respective voiding events, leakage, or any other event related to the patient. External device 24 may provide this user input to server 26.

One or more medical leads, e.g., lead 18, may be connected to IMD 16. Once it is determined that patient 14 is a candidate for implantation of IMD 16 (e.g., using techniques described above for FIG. 1) and lead 18, lead 18 may be surgically or percutaneously tunneled to place one or more electrodes carried by a distal end of the respective lead at a desired nerve or muscle site, e.g., one of the previously listed target therapy sites such as a tissue site proximate a spinal (e.g., sacral) or pudendal nerve. For example, lead 18 may be positioned such that electrodes 20 deliver electrical stimulation to a spinal, sacral or pudendal nerve to reduce a frequency and/or magnitude of contractions of bladder 12. Additional electrodes of lead 18 and/or electrodes of another lead may provide additional stimulation therapy to other nerves or tissues as well.

In the example shown in FIG. 2, lead 18 is cylindrical. Electrodes 20 may be ring electrodes, segmented electrodes, partial ring electrodes or any suitable electrode configuration. Segmented and partial ring electrodes each extend along an arc less than 360 degrees (e.g., 90-120 degrees) around the outer perimeter of lead 18. In some examples, segmented electrodes 20 of lead 18 may be useful for targeting different fibers of the same or different nerves to generate different physiological effects (e.g., therapeutic effects). In examples, lead 18 may be, at least in part, paddle-shaped (e.g., a “paddle” lead), and may include an array of electrodes on a common surface, which may or may not be substantially flat.

In some examples, one or more of electrodes 20 may be cuff electrodes that are configured to extend at least partially around a nerve (e.g., extend axially around an outer surface of a nerve). In some examples, one or more of electrodes 20 may be paddle electrodes. Delivering electrical stimulation via one or more cuff electrodes and/or segmented electrodes may help achieve a more uniform electrical field or activation field distribution relative to the nerve, which may help minimize discomfort to patient 14 that results from the delivery of electrical stimulation. An electrical field may define the volume of tissue that is affected when the electrodes 20 are activated. An activation field represents the neurons that will be activated by the electrical field in the neural tissue proximate to the activated electrodes.

An implanted IMD 16 may deliver electrical stimulation to provide a therapeutic effect that reduces or eliminates a dysfunctional state such as overactive bladder. The therapeutic effect may include an inhibitory physiological response related to voiding of patient 14, such as a reduction in bladder contraction frequency by a desired level or degree (e.g., percentage), a reduction in bladder afferent firing, altering a pelvic floor muscle/nerve response and/or status such as of the external urethral sphincter (EUS), levator ani nerve, external anal sphincter, and the like.

System 10 may also include an external device 24, as shown in FIG. 2. External device 24 may be an example of a computing device. In some examples, external device 24 may be a clinician programmer or patient programmer. In some examples, external device 24 may be a device for inputting information relating to a patient. In some examples, external device 24 may be a wearable communication device, with a therapy request input integrated into a key fob or a wristwatch, handheld computing device, smart phone, computer workstation, or networked computing device. External device 24 may include a user interface that is configured to receive input from a user (e.g., patient 14, a patient caretaker or a clinician). In some examples, the user interface includes, for example, a keypad and a display, which may for example, be a liquid crystal display (LCD) or light emitting diode (LED) display. In some examples, the user interface may include a turnable knob or a representation of a turnable knob. The keypad may take the form of an alphanumeric keypad or a reduced set of keys associated with particular functions. External device 24 may additionally or alternatively include a peripheral pointing device, such as a mouse, via which a user may interact with the user interface. In some examples, a display of external device 24 may include a touch screen display, and a user may interact with external device 24 via the display. It should be noted that the user may also interact with external device 24, server 26 and/or IMD 16 remotely via a networked computing device.

A user, such as a physician, technician, surgeon, electrophysiologist, or other clinician, may also interact with external device 24 or another separate programmer (not shown), such as a clinician programmer, to communicate with IMD 16 and/or server 26. Such a user may interact with external device 24 to retrieve physiological or diagnostic information from IMD 16. The user may also interact with external device 24 to program IMD 16, e.g., select values for the stimulation parameter values with which IMD 16 generates and delivers stimulation and/or the other operational parameters of IMD 16, such as magnitudes of stimulation energy, user requested periods for stimulation or periods to prevent stimulation, or any other such user customization of therapy. In some examples, the stimulation parameter values may be proposed by system 10, for example, by server 26 and a user may be able to accept or reject the stimulation parameter values. In other examples, the stimulation parameter values may be set by system 10, for example, by server 26. As discussed herein, the user may also provide input to external device 24 indicative of physiological events such as bladder fill level perception and void events.

In some examples, the user may use external device 24 to retrieve information from IMD 16 relating to the stimulation parameters. As another example, the user may use external device 24 to retrieve information from IMD 16 relating to evoked signals from patient tissue. In some examples, the user may retrieve information relating to the performance or integrity of IMD 16 or other components of system 10, such as lead 18 or a power source of IMD 16. In some examples, this information may be presented to the user as an alert if a system condition that may affect the efficacy of therapy is detected.

The user of external device 24 may also communicate with server 26. For example, the user of external device 24 may provide information relating to the patient to server 26, such as demographic information, medical history, lifestyle information, bladder events, level satisfaction with therapy or sensor data.

IMD 16 and external device 24 may communicate via wireless communication using any techniques known in the art. Examples of communication techniques may include, for example, Bluetooth, or low frequency or radiofrequency (RF) telemetry, but other techniques are also contemplated. In some examples, external device 24 may include a programming lead that may be placed proximate to the patient's body near the IMD 16 implant site in order to improve the quality or security of communication between IMD 16 and external device 24.

As described above, in FIG. 1, lead 102 includes electrodes 106A and 106B that are used to deliver stimulation and sense evoked signals respectively. In some examples, it may be possible that lead 18 is one example of lead 102. Accordingly, electrodes 106A and 106B may be each one of electrodes 20. Also, in some examples, it may be possible for IMD 16 to be stimulation device 114 of FIG. 1. For determining whether it is recommended for patient 14 to be implanted with IMD 16, processing circuitry (e.g., of stimulation device 114 or IMD 16) may be configured to sense evoked signals.

In accordance with one or more examples described in this disclosure, a physician may need to determine, prior to implantation of IMD 16, whether implantation of IMD 16 will provide effective therapy for patient 14. IMD 16, which may be an example of stimulation device 114, may remain outside of patient 14 during the evaluation, and lead 18 may be an evaluation needle or lead that is inserted into patient 14 near one or more nerves associated with pelvic disorder therapy of patient 14 to deliver one or more electrical stimulation pulses to the one or more nerves of via one or more inserted electrodes 20 (e.g., on a lead or formed on an evaluation needle), as described above with respect to FIG. 1.

In some examples, a percutaneous evaluation needle is used to stimulate patient 14 tissue and sense evoked signals. For example, the evaluation needle may be inserted through a foramen in the spine and positioned near patient 14 tissue for stimulation. In some examples, an introducer is inserted to allow the evaluation lead to be used. In some examples, the evaluation needle/lead may have a minimal profile to provide the least intrusive evaluation procedure to patient 14. For example, the evaluation needle or lead may include only one electrode of electrodes 20. In some examples, an exposed portion of the conductive material composing the evaluation needle may function as an electrode to deliver electrical stimulation and sense for evoked signals. IMD 16 may sense, using electrodes 20, one or more evoked signals generated by one or more signal sources in response to delivery of the one or more electrical stimulation pulses to the one or more nerves. Based on the one or more evoked signals, processing circuitry of IMD 16 may classify the patient into a therapy response group that indicates at least one of a therapy device type and a therapy program for delivering pelvic disorder therapy to patient 14.

That is, the processing circuitry of IMD 16 may determine, based at least in part on the evoked signal, a therapy response group of a plurality of therapy response groups for patient 14. In some examples, the therapy response group indicates whether implantation of IMD 16 is recommended. The processing circuitry may output information indicative of the determined therapy response group. For instance, the therapy response groups includes a first therapy response group for patients for whom implantation of IMD 16 is recommended, and the therapy response groups include a second therapy response group for patient for whom further evaluation is needed for whether implantation of IMD 16 is recommended.

As another example, there may be different types of IMD 16, such as rechargeable IMDs or non-rechargeable IMDs. A rechargeable IMD includes a rechargeable power source, and a non-rechargeable IMD includes a non-rechargeable power source. Based on the evoked signal (e.g., amplitude, frequency, etc.), the processing circuitry may determine whether a relatively high stimulation amplitude, pulse width, and/or frequency is needed for therapy (e.g., intermittent stimulation to preserve longevity of a power source). If relatively high stimulation amplitude, pulse width, and/or frequency is needed for therapy, the processing circuitry may determine that a rechargeable IMD is recommended (e.g., so that the power source can be periodically replenished from the drain of the high stimulation). If relatively high stimulation amplitude, pulse width, and/or frequency is not needed for therapy, the processing circuitry may determine that a non-rechargeable IMD is recommended.

Accordingly, in some examples, there may be three therapy response groups. The first therapy response group may be for patients for whom a rechargeable IMD is recommended, a second therapy response group may be for patients for whom a non-rechargeable IMD is recommended, and a third therapy response group may be for patients for whom further evaluation is needed to determine whether an IMD is recommended.

In some examples, IMD 16 delivers electrical therapy and sensing evoked signals using the evaluation needle. The evaluation needle may include one or more conductive portions at the tip that could serve as the stimulation and sensing electrode. The evoked signals sensed using the evaluation needle may indicate whether the placement of the needle, or a subsequent lead, would be sufficient to measure accurate evoked signals. In some examples, the sensed evoked signals (or features thereof) may indicate that stimulation parameters have been achieved such that stimulation is resulting in activation of patient tissue/nerves in a way known to be associated with a therapy response. In response to evoked signals sensed by the evaluation needle, a physician may alter the penetration depth, angle, or skin insertion location of the evaluation needle, or whether the evaluation needle is inserted on patient's 14 right or left side.

In some examples, rather than continue to use the evaluation needle to deliver electrical signals and sense evoked signals, the location determined as sufficient for sensing evoked signals may be set for insertion of the lead 18. The evaluation needle stylet may be removed, and lead 18 is placed through the foramen needle. The electrodes 20 of lead 18 may exit the tip of the foramen needle, contacting or resting near/adjacent to the desired patient tissue for electrical stimulation. After placing lead 18, the needle may be removed, and an introducer sheath or dilator may be inserted. A directional guide that assisted in placement of the needle may also be removed. In some examples, an introducer sheath may include an exposed metal portion for delivering stimulation pulses and sensing for evoked signals from patient tissue, as described with respect to lead 18 and/or the evaluation needle throughout this disclosure.

In order to classify patient 14 into a therapy response group (e.g., determine a therapy response group from a plurality of therapy response groups), processing circuitry of IMD 16 may determine that the evoked signal satisfies one or more criteria, i.e., that features of the evoked signal satisfy one or more criteria. In some examples, the one or more criteria may be stored in memory of IMD 16 and/or external device 24 or server 26. Again, in such examples, IMD 16 may not be implanted yet, and is effectively functioning as stimulation device 116 of FIG. 1. Also, it is possible that stimulation device 116 is used instead of IMD 16, where stimulation device 116 is a different type of stimulator than IMD 16.

In some examples, IMD 16 and/or external device 24 may be configured to determine one or more features of the sensed evoked signal (including examples where the sensed evoked signal is a composite signal). For example, IMD 16 and/or external device 24 may be configured to determine signal thresholds, peaks, peak amplitudes, number of peaks, areas under peaks, peak widths, time between peaks, ratios of peak amplitudes, widths, and/or areas, peak latency, signal valleys, valley amplitudes, number of valleys, areas above valleys, valley widths, time between valleys, ratios of valley amplitudes, widths, and/or areas, valley latency, root-mean-square signal value, signal skew, kurtosis, frequency and/or spectral content of the signal(s), Hjorth activity, Hjorth mobility, Hjorth complexity, or any other suitable signal feature. In some examples, IMD 16 and/or external device 24 may be configured to determine an amplitude of one or more peaks of the evoked signal that are greater than 1 millivolt (mV), or greater than 0.1 mV, or greater than 0.01 mV. In addition, signal might be measured at multiple amplitudes, and the growth curve with one of the above features can be utilized to estimate rate of growth of the signal, or muscle/neural threshold, or one or more inflection points.

In some examples, in response to determining that the evoked signal satisfies the one or more criteria, processing circuitry of IMD 16 may classify patient 14 into a therapy response group that indicates an implantable medical device as the therapy device type for delivering pelvic disorder therapy to patient 14, i.e., that patient 14 is a candidate for an implanted medical device for pelvic disorder therapy. In some examples, in response to determining that the evoked signal satisfies the one or more criteria, processing circuitry of IMD 16 may classify patient 14 into a therapy response group that indicates a battery type for an implanted medical device. For example, Processing circuitry of IMD 16 may classify patient 14 into a therapy response group that indicates patient 14 is a candidate for an implanted medical device with a rechargeable or non-rechargeable battery.

In some examples, in response to determining that the evoked signal satisfies the one or more criteria, processing circuitry of IMD 16 may classify patient 14 into a therapy response group that indicates a nerve for stimulation to deliver pelvic disorder therapy to patient 14. For example, processing circuitry of IMD 16 may determine that stimulating the current nerve near/adjacent to one or more of electrodes 20 is or is not effective in providing therapy to patient 14. IMD 16 may send a notification to external device 24 or other device in communication with IMD 16 to inform a physician to change nerves for stimulation.

IMD 16 and/or external device 24 may be configured to determine one or more classifications of the one or more determined features. For example, IMD 16 and/or external device 24 may be configured to execute a trained machine learning (ML) algorithm to determine whether therapy from an IMD will be effective or not for a patient. That is, the trained ML algorithm may classify patients into therapy response groups and use the classifications to predict therapy efficacy based on the determined classifications. In some examples, IMD 16 and/or external device 24 may be configured to output the evoked signals to an external device for processing, e.g., an external device, such as server 26, may execute the ML algorithm and communicate results to IMD 16 and/or external device 24.

FIG. 3 is a block diagram illustrating an example configuration of components of an IMD 200, in accordance with one or more techniques of this disclosure. IMD 200 may be an example of IMD 16 of FIG. 2. In the example shown in FIG. 3, IMD 200 includes stimulation generation circuitry 202, switch circuitry 204, sensing circuitry 206, communication circuitry 208, sensor(s) 222, power source 224, lead 230 carrying electrodes 232, which may correspond lead 18 electrodes 20 of FIG. 2. In the example shown in FIG. 3, IMD 200 includes processing circuitry 210 and storage device 212. Processing circuitry 210 may include one or more processors configured to perform various operations of IMD 200.

In the example shown in FIG. 2, storage device 212 store stimulation parameter settings 242. In addition, storage device 212 may store evoked signal data 254 obtained directly or indirectly from one or more electrodes 232 and/or sensors 222. In this case, IMD 200 of FIG. 3 may process evoked signal data 254 and select or adjust stimulation parameter settings 242, including cycling, based on the evoked signal data 254.

Evoked signal data 254 may include sensed signals from one or more signal sources (e.g., which may be stimulation-evoked and referred to as stimulation-evoked signals) and/or sensed composite stimulation-evoked signals, such as those described above. In some examples, evoked signal data 254 may include raw sensed signals from sensor(s) 222 and/or amplified, filtered, and/or analog-to-digital converted signals, e.g., via sensing circuitry 206. For example, evoked signal data 254 may include a time-varying signal indicative of a response or responses of one or more signal sources (e.g., nerves and/or muscles) to electrical stimulation, such as illustrated and described below with reference to FIGS. 5-9. In some examples, evoked signal data 254 may include an averaged signal and/or one or more signal features determined via processing of the signal, e.g., peak/valley detection, peak/valley amplitude, width, and/or area, frequency analysis, digital signal processing, signal latency, and the like. In some examples, the one or more signal features may include one or more of a signal peak, a signal peak amplitude, a number of signal peaks, an area under one or more signal peaks, a signal peak width, a time between signal peaks, a ratio of signal peak amplitudes, a ratio of signal peak widths, a ratio of areas under signal peaks, a latency of a signal peak, a signal valley, a signal valley amplitude, a number of signal valleys, an area above one or more signal valleys, a signal valley width, a time between signal valleys, a ratio of signal valley amplitudes, a ratio of signal valley widths, a ratio of areas above signal valleys, a valley latency, a root-mean-square signal value, a signal skew, a signal kurtosis, a signal frequency, a signal spectral content, a Hjorth feature, a signal amplitude growth curve threshold, a signal amplitude growth curve inflection point amplitude, a signal amplitude growth curve inflection point latency, a signal amplitude growth curve saturation point, a signal strength duration curve chronaxie, a signal strength duration curve rheobase, or another signal strength duration curve feature, a signal maximum rate of change feature (e.g., maximum of the derivative of the signal), or a signal minimum rate of change feature (e.g., the minimum of the derivative of the signal), or any other suitable signal feature. In some examples, evoked signal data 254 may include additional information, such as sensor(s) 222 settings during sensing of evoked signals, a timestamp denoting the date and/or time one or more evoked signals are sensed, patient information including a current physiological state of patient 14 physiological measurements of patient 14 at or near the time one or more evoked signals are sensed, e.g., heart rate, temperature, blood pressure, patient activity, motion, and/or posture (e.g., patient input and/or measured, such as from a patient smartphone, wearable device, external device 24 or IMD 200, or other device) and the like, or patient input such as a pain level and/or pain score, voiding and/or voiding frequency, patient medical history information, patient age or other demographic information, or any other suitable patient input information.

In one or more examples, IMD 200 may not store or receive the evoked signal data 254. Instead, external device 24 or another device may directly or indirectly select or adjust stimulation parameter settings based on evoked signal data 254 and communicate the selected settings or adjustments to IMD 200. In some examples, stimulation parameter settings 242 may include stimulation parameters (sometimes referred to as “sets of therapy stimulation parameters”) for respective different stimulation programs selectable by the clinician or patient for therapy. In some examples, stimulation parameter settings 242 may include one or more recommended parameter settings. In this manner, each stored therapy stimulation program, or set of stimulation parameters, of stimulation parameter settings 242 defines values for a set of electrical stimulation parameters (e.g., a stimulation parameter set), such as electrode combination (selected electrodes and polarities), stimulation current or voltage amplitude, stimulation pulse width, and pulse frequency.

In some examples, stimulation parameter settings 242 may indicate for the stimulation to turn on for a certain period of time, and/or to turn off stimulation for a certain period of time. For example, stimulation parameter settings 242 may further include cycling information indicating when or how long stimulation is turned on and off, e.g., periodically and/or according to a schedule. For example, electrical stimulation may be delivered as a series of electrical stimulation pulses, each pulse being defined by an amplitude, a frequency, a pulse width and/or duration, and an electrical combination (e.g., stimulation pulse parameters). Cycling parameters may define how the series of pulses is delivered. For example, stimulation cycling parameters may include a cycling frequency or period and a duty cycle or ratio of how long electrical stimulation pulses are delivered according to the cycling frequency (an “on-time”) to how long electrical stimulation is not delivered (an “off-time). In other examples, cycling may include a schedule defining the specific times at which electrical stimulation pulses are to be delivered according to specific stimulation pulse parameter settings.

In some examples, an electrical stimulation pulse may comprise electrical stimulation delivered according to one or more electrical stimulation parameter settings 242, e.g., electrical stimulation delivered according to stimulation pulse parameters settings, stimulation cycling parameters settings, and/or any other suitable stimulation parameters settings, information, limits, or conditions.

Stimulation generation circuitry 202 includes electrical stimulation circuitry configured to generate electrical stimulation and generates electrical stimulation pulses selected to alleviate symptoms of one or more diseases, disorders or syndromes. While stimulation pulses are described, stimulation pulses may take other forms, such as continuous-time signals (e.g., sine waves) or the like. The electrical stimulation circuitry may reside in an implantable housing, for example of the IMD. Lead 230 may include any number of electrodes 232. The electrodes are configured to deliver the electrical stimulation to the patient. In the example of FIG. 3, electrodes 232 includes eight electrodes A-H. In some examples, IMD 200 is a trialing device, and kept external, to evaluate whether a patient is a candidate for therapy, and IMD 200 may only include a single lead or evaluation needle (e.g., such as where IMD 200 is stimulation device 114 in FIG. 1) with a single electrode, or possibly one electrode for stimulation and one electrode for sensing, as illustrated in FIG. 1. However, if determined that IMD 200 is to be implanted, then IMD 200 may be coupled to lead 230 and implanted.

As described with respect to FIG. 1, although not illustrated, in some examples, lead 230 may include one or more reference electrodes (e.g., ground electrode). The one or more reference electrodes may provide a return path for the stimulation pulses from an electrode on lead 230, or may provide a reference for signals sensed by another electrode on lead 230. In some examples, in addition to or instead of reference electrodes on lead 230, IMD 200 may include the reference electrodes (e.g., such as on the housing of IMD 200). Other examples of reference electrodes are possible such as reference electrodes attached to the skin of patient 14.

In some examples, the electrodes are arranged in bipolar combinations. A bipolar electrode combination may use electrodes carried by the same lead 230 or different leads. Switch circuitry 204 may include one or more switch arrays, one or more multiplexers, one or more switches (e.g., a switch matrix or other collection of switches), or other electrical circuitry configured to direct stimulation pulses from stimulation generation circuitry 202 to one or more of electrode 232, or directed sensed signals from one or more of electrodes 232 to sensing circuitry 206. In some examples, each of the electrodes 232 may be associated with respective regulated current source and sink circuitry to selectively and independently configure the electrode to be a regulated cathode or anode. Stimulation generation circuitry 202 and/or sensing circuitry 206 also may include sensing circuitry to direct electrical signals sensed at one or more of electrodes 232.

Sensing circuitry 206 may be configured to monitor signals from any combination of electrodes 232 and/or sensor(s) 222. In some examples, sensing circuitry 206 includes one or more amplifiers, filters, and analog-to-digital converters. Sensing circuitry 206 may be used to sense evoked and/or physiological signals, such as ECAP signals, EMG signals, and the like. Sensing circuitry 206 may provide signals to an analog-to-digital converter, for conversion into a digital signal for processing, analysis, storage, or output by processing circuitry 210. In some examples, sensing circuitry 206 may sense and/or detect stimulation-evoked signals and/or composite stimulation-evoked signals comprising one or more of an ECAP, an EMG or surface EMG, an MMG, a network excitability, and/or multiple signals of differing signal type evoked by one or more signal sources such as sacral nerves and their branches, e.g., dorsal and ventral rami of sacral nerves, pudendal nerves, sciatic nerves, saphenous nerves, nerves in the sacral plexus, pelvic nerves, pelvic plexus nerves, pelvic splanchnic nerves, inferior hypogastic plexus nerves, lumbosacral trunk nerves, e.g., where the lumbosacral trunk joins sacral nerves, any sympathetic nerve fibers in the sympathetic chain of any of the above nerves or other nerves, muscles such as an external anal sphincter muscle, coccygeus muscle, levator ani muscle group, bulbocavernosus and/or bulbospongiosus muscle, gluteal muscles, e.g., gluteal maximus, gluteal medius, and gluteal minimus, perineal muscles, ischiocavernosus muscles, puborectalis muscles, piriformis muscles, or any other muscles.

Sensor(s) 222 may be configured to sense one or more physiological responses of a patient, e.g., patient 14. In some examples, sensors 222 may be other sensors located at one or more other positions on patient 14, located at or near one or more muscles and or nerves, or located at positions on patient 14 which may be relatively far from a signal source, e.g., a nerve or muscle.

Communication circuitry 208 supports wireless communication between IMD 200 and an external programmer or another computing device under the control of processing circuitry 210. Processing circuitry 210 of IMD 200 may receive, as updates to programs, values for various stimulation parameters such as amplitude and electrode combination, from the external programmer via communication circuitry 208. Processing circuitry 210 of IMD 200 may store updates to the stimulation parameter settings 242 or any other data in storage device 212. Communication circuitry 208 in IMD 200, as well as communication circuits in other devices and systems described herein, such as the external programmer and patient feedback sensing system, may accomplish communication by Bluetooth or radiofrequency (RF) communication techniques. In addition, communication circuitry 208 may communicate with an external medical device programmer via proximal inductive interaction of IMD 200 with the external programmer, where the external programmer may be one example of external device 24 of FIG. 1. Accordingly, communication circuitry 208 may send information to the external programmer on a continuous basis, at periodic intervals, or upon request from IMD 16 and/or external device 24.

Processing circuitry 210 may include one or more processors, such as any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software or any combination thereof. Processing circuitry 210 controls stimulation generation circuitry 202 to generate stimulation pulses according to stimulation parameter settings 242. In some examples, processing circuitry 210 may execute other instructions stored in storage device 212 to apply stimulation parameters specified by one or more of programs, such as electrode combination or configuration, electrode polarity, amplitude, pulse width, pulse shape, pulse frequency or pulse rate, or cycling of each of the stimulation pulses.

In the illustrated example of FIG. 2, processing circuitry 210 includes a signal unit 216 to process evoked signals. Signal unit 216 may represent an example of a portion of processing circuitry configured to process evoked signals.

Storage device 212 may be configured to store information within IMD 200 during operation. Storage device 212 may include a computer-readable storage medium or computer-readable storage device. In some examples, storage device 212 includes one or more of a short-term memory or a long-term memory. Storage device 212 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, storage device 212 is used to store data indicative of instructions, e.g., for execution by processing circuitry 210, respectively. As discussed above, storage device 212 is configured to store stimulation parameter settings 242.

Power source 224 is configured to deliver operating power to the components of IMD 200. Power source 224 may include a battery and a power generation circuit to produce the operating power. In some examples, the battery is rechargeable to allow extended operation. In some examples, recharging is accomplished through proximal inductive interaction between an external charger and an inductive charging coil within IMD 200. Power source 224 may include any one or more of a plurality of different battery types, such as nickel cadmium batteries and lithium ion batteries.

In some examples, processing circuitry 210 of IMD 200 directs delivery of electrical stimulation by the electrodes 232, receives evoked signal data and/or information from sensors 222, and generates output based on the received data and/or information. The signal unit 216 may use evoked signal data to classify the patient into a therapy response group, wherein the therapy response group indicates a therapy device type for delivering pelvic disorder therapy to the patient. That is, signal unit 216 may be configured to determine, based at least in part on the evoked signal, a therapy response group of a plurality of therapy response groups for the patient, wherein the therapy response group indicates whether implantation of the implantable medical device is recommended, and output information indicative of the determined therapy response group.

Processing circuitry 210 may control stimulation circuitry 202 to deliver stimulation energy with stimulation parameters specified by one or more stimulation parameter settings 242 stored on storage device 212 and, to collect evoked signals pertaining to the stored stimulation parameter settings 242. Processing circuitry 210 collects this evoked signal information by receiving the information via sensing circuitry 206 and/or sensors 222. Processing circuitry 210 may also control stimulation circuitry 202 to test different parameter settings and record one or more corresponding evoked signals for each selected combination, and test different parameter settings as they compare to one or more sensed evoked signals. For example, processing circuitry 210 directs stimulation circuitry 202 to deliver stimulation via a particular cycling and the signal unit 216 collects the corresponding evoked signal data 254 from sensing circuitry 206. The evoked signal data 254 for this test may be stored in the storage device 212.

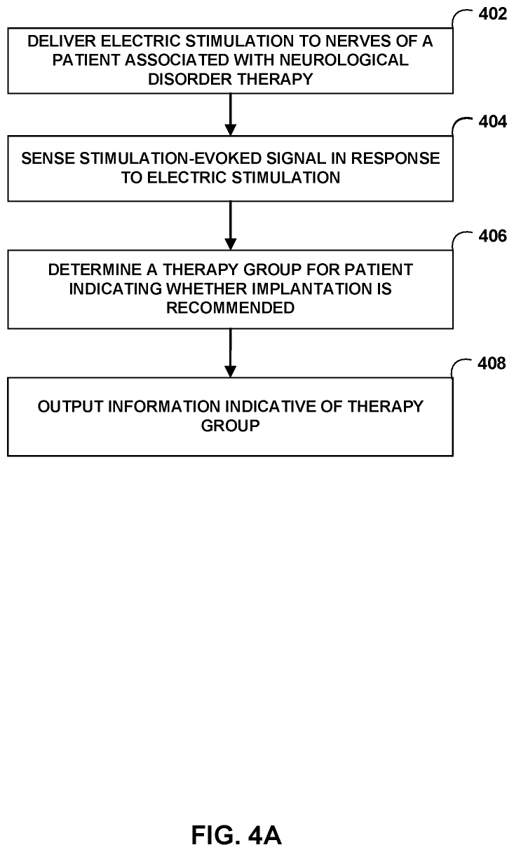

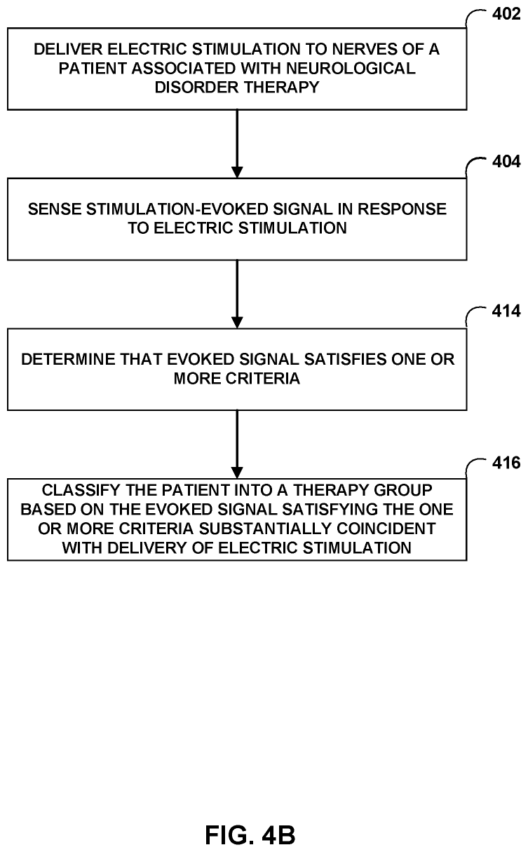

FIG. 4A is a flow diagram illustrating an example method of determining a therapy response group for a patient, in accordance with one or more techniques of this disclosure. Although FIG. 4A is discussed with respect to system 100 of FIG. 1, it is to be understood that the methods discussed herein may include and/or utilize other systems and methods in other examples.

Processing circuitry of stimulation device 114 may deliver (e.g., via stimulation circuitry of stimulation device 114) one or more electrical stimulation pulses to one or more nerves 112 of patient 14 via one or more inserted electrodes (e.g., electrodes 106) and prior to implantation of an implantable medical device, wherein the one or more nerves are associated with neurological disorder therapy (402). For example, the neurological disorder therapy may be pelvic disorder therapy. For example, processing circuitry of stimulation device 114 may control stimulation circuitry of stimulation device 114 to deliver stimulation energy via one or more of electrodes 106 with stimulation parameters specified by one or more stimulation parameter settings stored on a storage device. In some examples, the electrical stimulation pulse may be delivered to one or more of a sacral nerve, a sacral nerve branch, a saphenous nerve, a sciatic nerve, a tibial nerve, a pelvic nerve, or a pudendal nerve in any combination. In other examples, the electrical stimulation pulse may be delivered to any other nerve or muscle, any portion of the patient's brain, any organ of the patient, or any other tissue of the patient. In some examples, the one or more nerves may be associated with incontinence therapy. In some examples, stimulation device 114 delivers the one or more electrical stimulation pulses via an evaluation lead. In some examples, stimulation device 114 delivers the one or more electrical stimulation pulses via an evaluation needle.

Processing circuitry of stimulation device 114 may sense (e.g., via sensing circuitry of stimulation device 114), via one or more of electrodes 106 may sense an evoked signal generated by one or more signal sources in response to delivery of the one or more electrical stimulation pulses (404). For example, the processing circuitry may control stimulation circuitry, communication circuitry, and/or sensing circuitry of stimulation device 114 to collect evoked signal information, e.g., stimulation-evoked signal data. In some examples, processing circuitry senses a composite stimulation-evoked signal comprising a composite of signals generated by two or more signal sources in response to the one or more electrical stimulation pulses. For example, a composite stimulation-evoked signal sensed by sensing circuitry may be a composite of a plurality of stimulation-evoked signals, each of which may originate from a different signal source (e.g., muscle, nerve, etc.), each of which may originate at the same time or at a different time, and each of which may have the same or different duration. The processing circuitry may store received evoked signal data in a storage device of stimulation device 114. In some examples, stimulation device 114 may receive evoked signal(s) as one or more of physiological signals. For example, stimulation device 114 may receive one or more ECAP, EMG, MMG, and the like. In some examples, the processing circuitry may deliver the electrical stimulation pulses and sense the evoked signals using the same electrode of electrodes 106.

The processing circuitry of stimulation device 114 may determine, based at least in part on the evoked signal, a therapy response group of a plurality of therapy response groups for patient 14, wherein the therapy response group indicates whether implantation of the IMD is recommended for delivering pelvic disorder therapy to patient 14 (406). In some examples, determining the therapy response group for patient 14 may include classifying the patient into a therapy response group that indicates whether implantation of the IMD is recommended. In some examples, stimulation device 114 is an evaluation device to determine if patient 14 is a candidate for implantation of an IMD for pelvic disorder therapy. A physician may need to determine, prior to implantation of an IMD, whether implantation of the IMD will provide effective therapy for patient 14. A therapy response group may indicate one or more different device types for providing effective therapy for patient 14, for example an implantable medical device, a rechargeable device, and/or a non-rechargeable device.

In some examples, based on the evoked signal, processing circuitry of stimulation device 114 may determine the therapy response group for the patient by classifying patient 14 into a therapy response group that indicates that implantation of the implantable medical device is recommended for delivering pelvic disorder therapy to patient 14, i.e., that patient 14 is a candidate for an implanted medical device as the device type for pelvic disorder therapy. In some examples, based on the evoked signal, processing circuitry of stimulation device 114 may classify patient 14 into a therapy response group that indicates a battery type for an implanted medical device. For example, processing circuitry of stimulation device 114 may classify patient 14 into a therapy response group that indicates patient 14 is a candidate for an implanted medical device with a rechargeable or non-rechargeable battery. In some examples, based on the evoked signal, processing circuitry of stimulation device 114 may classify patient 14 into a therapy response group that indicates a nerve for stimulation to deliver pelvic disorder therapy to patient 14. For example, processing circuitry of stimulation device 114 may determine that stimulating the current nerve near/adjacent to one or more of electrodes 106 is or is not effective in providing therapy to patient 14. stimulation device 114 may send a notification to an external device or other device in communication with stimulation device 114 to inform a physician to change nerves for stimulation.

The processing circuitry of stimulation device 114 may classify patient 14 into a therapy response group substantially coincident with the delivery of the one or more electrical stimulation pulses. For example, the processing circuitry may classify patient 14 into a therapy response group in a time period after delivery of the one or more electrical stimulation pulses. In some examples, the time period may be less than one day, less than one hour, less than one minute, and/or less than one second. By classifying patient 14 into a therapy response group within such a short time period, system 100 may allow a physician to determine if patient 14 is a candidate for therapy by an IMD in a single out-patient procedure, rather than requiring patient 14 to wear a trialing lead for days to weeks.

The processing circuitry of stimulation device 114 may output information indicative of the determined therapy response group (408). For example, stimulation device 114 may include a display for displaying information, including whether implantation of an IMD is recommended for patient 14. In some examples, stimulation device 114 may be in communication with an external device (e.g., external device 24 of FIG. 2, or a computing device of server 26 of FIG. 2), and processing circuitry of stimulation device 114 may send information to the external device indicating a determined therapy response group for patient 14. For example, the processing circuitry may send information in the form of text or imagery to be displayed on a user interface of the external device indicating the determined therapy response group for patient 114. In some examples, the external device may be a patient or clinician programmer.

FIG. 4B is a flow diagram illustrating another example method of determining a therapy response group for a patient, in accordance with one or more techniques of this disclosure. Although FIG. 4B is discussed using stimulation device 114 of FIG. 1, it is to be understood that the methods discussed herein may include and/or utilize other systems and methods in other examples.

Processing circuitry of stimulation device 114 may deliver one or more electrical stimulation pulses as described above with reference to FIG. 4A (402). In addition, processing circuitry of stimulation device 114 may sense one or more evoked signals as described above with reference to FIG. 4A (404).

The processing circuitry of stimulation device 114 may determine whether the one or more evoked signals satisfy one or more criteria (414). In order to classify patient 14 into a therapy response group (e.g., determine the therapy response group) substantially coincident with the delivery of the one or more electrical stimulation pulses, the processing circuitry may be configured to determine whether the evoked signal satisfies the one or more criteria. For example, the evoked signal may be sensed or recorded by stimulation device 114 as signal data, which may include a time-varying signal indicative of a response or responses of one or more signal sources (e.g., nerves and/or muscles) to the electrical stimulation, discussed in more detail below with reference to FIGS. 5-9. For example, IMD 16 and/or external device 24 may be configured to determine signal thresholds, peaks, peak amplitudes, number of peaks, areas under peaks, peak widths, time between peaks, ratios of peak amplitudes, widths, and/or areas, peak latency, signal valleys, valley amplitudes, number of valleys, areas above valleys, valley widths, time between valleys, ratios of valley amplitudes, widths, and/or areas, valley latency, root-mean-square signal value, signal skew, kurtosis, frequency and/or spectral content of the signal(s), Hjorth activity, Hjorth mobility, Hjorth complexity, or any other suitable signal feature. The criteria may include a threshold number of peaks present in the signal, a peak amplitude threshold, a threshold number of peaks that exceed a threshold amplitude, a threshold area under the curve of the evoked signal data, etc.

In some examples, the one or more criteria may be stored in a memory of stimulation device 114 and/or another computing device in communication with stimulation device 114. In some examples, criteria for the evoked signal data may be adaptive, and change based on a database of received and/or stored evoked signal data. For example, a computing device (e.g., server 26 of FIG. 1) in communication with a plurality of stimulation devices may be configured to collect and store a plurality of evoked signal data from a plurality of patients, and correlate the plurality of evoked signal data with therapy response groups and/or therapy outcomes for the respective patients to whom the evoked signal data correlates.

In some examples, the computing device may determine the one or more criteria for the evoked signal based on a trained machine learning model. For example, the computing device may collect a plurality of patient therapy data from a plurality of patients. The plurality patient therapy data may include evoked signal data from the plurality of patients undergoing known therapy programs (e.g., with known therapy devices, known therapy settings, and/or known nerves for stimulation). The computing device may train the machine learning model using evoked signal data as input, and known therapy programs as output. The computing device may be configured to periodically retrain the machine learning model over time (e.g., every six months) or when prompted by a user of the computing device. The computing device may retrain the machine learning model using patient therapy data (e.g., evoked signal data and known patient therapy programs) collected since the previous training of the machine learning model. In some examples, the machine learning model may be stored in memory of stimulation device 114.

Processing circuitry may, responsive to determining that the evoked signal satisfies the one or more criteria, classify patient 14 into a therapy response group substantially coincident with delivery of the electric stimulation (416). In some examples, the processing circuitry may or may not classify patient 14 into a therapy response group based on whether the evoked signal satisfies the one or more criteria.

In some examples, in response to determining that the evoked signal does not satisfy one or more criteria, the processing circuitry may classify patient 14 into a therapy response group that indicates the patient is a candidate for a longer term evaluation of the candidacy of patient 14 for pelvic disorder therapy. Longer term evaluation of the candidacy of the patient may include multiple-day determinations of the efficacy of pelvic disorder therapy, e.g., by way of a stimulation trialing device that is not implanted or a trialing device with an implanted lead but an external device for stimulation generation. For example, the processing circuitry may classify patient 14 into a therapy response group that indicates that processing circuitry is unable to determine whether full implantation of an IMD for pelvic disorder therapy would be effective for patient 14.

In some examples, in response to determining that the evoked signal does not satisfy one or more criteria, the processing circuitry may classify patient 14 into a therapy response group that indicates the patient is not a candidate for pelvic disorder therapy. For example, processing circuitry may classify patient 14 into a therapy response group that indicates patient 14 would not benefit significantly from therapy provided by an implanted IMD.

In some examples, in response to determining that the evoked signal satisfies one or more criteria, the processing circuitry may classify patient 14 into a therapy response group that indicates a nerve of the patient to provide pelvic disorder therapy. For example, the processing circuitry may classify the patient into a therapy response group that indicates that stimulation of a sacral nerve would be an effective stimulation nerve for pelvic disorder therapy. In some examples, the processing circuitry may classify the patient into a therapy response group that indicates that stimulation of a tibial nerve would be an effective stimulation nerve for pelvic disorder therapy. In some examples, the processing circuitry may classify the patient into a therapy response group that indicates that stimulation of muscle tissue would be effective for pelvic disorder therapy. In some examples, the processing circuitry may classify the patient into a therapy response group that indicates that stimulation of a certain part of a nerve would be effective for pelvic disorder therapy. In some examples, the processing circuitry may classify the patient into a therapy response group that indicates a particular branch of a sacral nerve would be an effective stimulation nerve for pelvic disorder therapy. In some examples, the processing circuitry may classify the patient into a therapy response group that indicates nerves on a left side or a right side of the patient as effective stimulation nerves for pelvic disorder therapy.

In some examples, the processing circuitry may indicate that it cannot classify patient 14 into a therapy response group based on the evoked signal received from the current stimulation location of one or more of electrodes 106. The processing circuitry may indicate that a location of lead 102 and one or more of electrodes 106 should change in order to better classify patient 14 into a therapy response group. For example, the processing circuitry may deliver a first set of one or more electrical stimulation pulses to nerves 112 of patient 14 via one or more of electrodes 106 located at a first location within patient 14. The processing circuitry may then sense a first evoked signal generated by nerves 112 at the first location in response to the first set of one or more electrical stimulation pulses. The processing circuitry may determine that the first evoked signal does not satisfy one or more criteria. The processing circuitry may then deliver a second set of one or more electrical stimulation pulses to nerves 112 or other nerves of patient 14 at a second location within patient 14. The processing circuitry may then determine if a second evoked signal from delivery of the second set of one or more electrical stimulation pulses satisfies one or more criteria. The processing circuitry may classify patient 14 into a therapy response group based at least in part on whether the second set of one or more electrical stimulation pulses satisfies the one or more criteria. In some examples, this procedure may repeat until processing circuitry is able to classify the patient into a therapy response group. In between delivery of sets of electrical stimulation pulses, a physician may relocate one or more of electrodes 106 and/or lead 102.

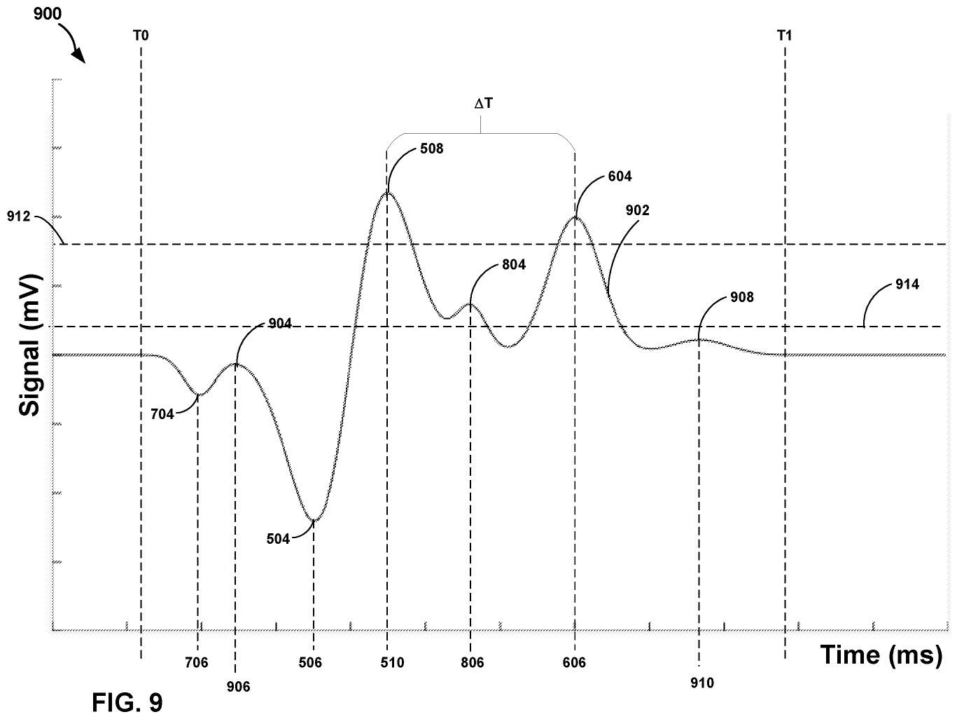

FIGS. 5-8 are plots of example evoked signals (e.g., stimulation-evoked signals) and FIG. 9 is an example composite evoked signal, and are described together below. In the specific examples of FIGS. 5-9 below, each signal plotted represent a voltage amplitude of a circuit including an electrode that varies in time in proportion to a time-varying electric field sensed by the electrode. The time-varying field in the examples shown is caused by one or more signal sources, e.g., nerve, muscle, or other tissue, of a patient in response to electrical stimulation. However, FIGS. 5-9 may generally represent one or more other quantities. In some examples, each signal plot may represent an amplitude as a function of time of a sensed quantity over time, the quantity varying in proportion to a physiological response of a signal source. In some examples, the quantity is an amplitude measured by a sensor. For example, the amplitude may be a voltage and/or current that varies in time according to an amplitude of an electric field and/or potential emitted and/or induced by a signal source. In some examples, composite evoked signal 902 described below may be a composite of sensed quantities from a plurality of sources sensed by one or a plurality of sensors, e.g., combined amplitudes as a function of time from two or more different sensors sensing two or more different quantities from one or more different signal sources that respond to the same electrical stimulation at or near the same time or within a period of time (e.g., a sensing “time window”). In some examples, a single sensor may include a single electrode on an inserted lead, or an exposed conductive tip of an evaluation needle configured to both provide electrical stimulation and sensed for an evoked signal. In some examples, two sensors may sense two different quantities from the same signal source, e.g., an EMG and an MMG of a muscle response. In other examples, composite evoked signal 902 may be a composite of a sensed quantity, e.g., an electric field and/or potential, from a plurality of signal sources sensed by the same sensor, e.g., an electrode sensing a varying electric field that is a superposition of a plurality of electric fields caused by a plurality of signal sources responding to electrical stimulation within a sensing time window.

Although specific examples of processing circuitry classifying a patient into specific therapy response groups are provided with reference to different figures below based on different criteria, it is understood that the processing circuitry may classify the patient into any therapy response group given satisfaction or nonsatisfaction of any criteria.

FIG. 5 is a plot 500 of an example evoked signal 502, in accordance with one or more techniques of this disclosure. In the example shown, signal 502 is a voltage amplitude that varies in time in proportion to a time-varying electric field sensed by an electrode, the time-varying electric field caused by a signal source in response to electrical stimulation. In the example shown, time T0 corresponds to a time at which electrical stimulation of a nerve or muscle ceases, e.g., is turned off, and time T1 corresponds to the ending time of the sensing time window, e.g., the sensing time window is the difference between T0 and T1. In some examples, signal 502 may have a signal length in time that is equal to the time window, e.g., the physiological response of the signal source emits a detectable quantity (e.g., electric field) that lasts for the length of the time window. In other examples, the signal length of signal 502 may be less than the time window. Generally, the time window may be chosen based on signal length, e.g., time T0 may be chosen to be the time at which electrical stimulation ceases and time T1 may be chosen based on the time-length of the sensed signal, e.g., any of 502, 602, 702, 802, and/or 902. In the examples of FIGS. 5-9, T1 chosen based on an exemplary time-length of signal 902 and is shown on each of plots 500-900 for reference. In some examples, the length of evoked signals 502-902 may be relatively long, e.g., 5 ms, 10 ms, 15 ms, 20 ms, 30 ms, or longer. In some examples, the shape, length, and location along the time axis of one or more features of evoked signals 502-802 may be different.

Processing circuitry of a stimulation device may determine one or more criteria for evoked signal 502. For example, the criteria may include one or more of determining whether one or more peaks in evoked signal 502 exceeds a threshold, determining whether a number of peaks in evoked signal 502 exceeds a threshold, determining whether an area under the curve of evoked signal 502, determining whether evoked signal 502 exceeds a threshold, determining whether a peak is present in a latency window of evoked signal 502, etc. The processing circuitry may further classify the patient into a therapy response group based on evoked signal 502 satisfying the one or more criteria.

In the example shown, signal 502 includes valley 504 (which may be considered a “peak” with a negative amplitude and may be simply referred to as a “peak” herein) at time 506 and peak 508 at time 510. In the example shown, signal 502 may be a stimulation-evoked signal of a neural response of certain fibers of a nerve to electrical stimulation.

In some examples, criteria for the evoked signal data may include the presence of a peak within a latency window of evoked signal 502. The criteria may further include the presence of a peak with positive amplitude, and/or the presence of a peak with negative amplitude. The example shown in FIG. 5 includes peak 508 with a positive amplitude and peak 504 with a negative amplitude. The example in FIG. 5 may satisfy the criteria that there be a peak within the evoked signal data. For example, in response to determining that evoked signal 502 in FIG. 5 contains peak 508, processing circuitry of a stimulation device may classify the patient into a therapy response group that indicates the current nerve/nerve location being stimulated as the nerve for therapy.

In some examples, criteria for the evoked signal data may include the amplitude of a peak in evoked signal 502 exceeding a threshold amplitude. In the example of FIG. 5, peak 508 exceeds peak threshold 512, and may satisfy the criteria for exceeding peak threshold 512.

FIG. 6 is a plot 600 of another example evoked signal 602, in accordance with one or more techniques of this disclosure. In the example shown, signal 602 is a voltage amplitude that varies in time in proportion to a time-varying electric field sensed by an electrode, the time-varying electric field caused by a signal source in response to electrical stimulation. In the example shown, signal 602 includes peak 604 at time 606. In some examples, signal 602 may be a stimulation-evoked signal of an EMG of a muscle in response to electrical stimulation.

In some examples, criteria for the evoked signal data may include a threshold area under the curve of evoked signal 602. The criteria may further include positive and/or negative area under the curve of evoked signal 602. The example shown in FIG. 6 includes area under the curve 612. The example in FIG. 6 may satisfy the threshold for a positive area under the curve of evoked signal 602. For example, in response to determining that area under the curve 612 of evoked signal 602 in FIG. 6 satisfies the threshold area under the curve criteria, processing circuitry of the stimulation device may classify the patient into a therapy response group that indicates an implantable IMD as a therapy device for pelvic disorder therapy.

FIG. 7 is a plot 700 of another example evoked signal 702, in accordance with one or more techniques of this disclosure. In the example shown, signal 702 is a voltage amplitude that varies in time in proportion to a time-varying electric field sensed by an electrode, the time-varying electric field caused by a signal source in response to electrical stimulation. In the example shown, signal 702 includes valley 704 at time 706. In the example shown, signal 702 may be a stimulation-evoked signal of a neural response of nerve fibers to electrical stimulation.

In some examples, the processing circuitry may determine that evoked signal 702 does not satisfy a peak amplitude threshold. In response to determining that evoked signal 702 does not satisfy the peak amplitude threshold, processing circuitry may classify the patient into a therapy response group that indicates that the device type is a device with or without a rechargeable battery.

FIG. 8 is a plot 800 of another example evoked signal 802, in accordance with one or more techniques of this disclosure. In the example shown, signal 802 is a voltage amplitude that varies in time in proportion to a time-varying electric field sensed by an electrode, the time-varying electric field caused by a signal source in response to electrical stimulation. In the example shown, signal 802 includes peak 804 at time 806. In the example shown, signal 802 may be a stimulation-evoked signal of a neural response of one or more fibers of a nerve or an EMG of a muscle in response to electrical stimulation.

In some examples, the processing circuitry may determine that evoked signal 802 does not satisfy a peak amplitude threshold. In response to determining that evoked signal 802 does not satisfy the peak amplitude threshold, processing circuitry may classify the patient into a therapy response group that indicates that the patient should undergo an elongated evaluation period.

FIG. 9 is a plot of an example composite evoked signal, in accordance with one or more techniques of this disclosure. In the example shown, signal 902 is a voltage amplitude that varies in time in proportion to a time-varying electric field sensed by an electrode, the time-varying electric field caused by a plurality of signal sources in response to electrical stimulation. For example, signal 902 may be a composite of signals 502-802. Although not shown, signal 902 may include other peaks, features, artifacts, and/or noise. For example, electrode may sense signal 902 but not signals 502-802, which are illustrated for as individual components of composite signal 902 for clarity.

In the example shown, composite evoked signal 902 includes peaks 504, 508, 604, 704, 804, and 904 and 908 occurring at times 506, 510, 606, 706, 806, and 906 and 910, respectively. In the example shown, peak 904 may correspond to a combination of two or more signal sources. In other words, peak 904 may not be a peak caused by a signal source, but rather is a result of the combination of signals 502 and 702. Peak 908 may be a stimulation-evoked signal of an EMG of a muscle in response to electrical stimulation, e.g., a second contraction of the same muscle of peak 604 or a different muscle.

In some examples, a plurality of features of signal 902 may be determined, e.g., per block 404 of the method illustrated and described above with reference to FIGS. 4A and 4B. For example, stimulation device 114 or another device such as a computing device, may determine/receive signal 902 and determine one or more peaks 504, 508, 604, 704, 804, 904 and 908, the corresponding times of the peaks, latency between one or more peaks such as DT between peak 508 and 604, the widths and areas of any of the above peaks, the frequency and/or spectral content of signal 902, or any other signal feature, e.g., derivable via signal processing and/or digital signal processing.

In some examples, one or more determined feature may correspond to, and may be correlated with, the efficacy of stimulation therapy. For example, peak 504 may relate to an electrical stimulation response of certain fibers of a nerve to electrical stimulation, peak 604 may relate to an EMG of a muscle, and peak 704 may relate to an electrical stimulation response of nerve fibers, e.g., which may relate to sensory and motor information. In some examples, improved and or optimal electrical stimulation therapy may be electrical stimulation that excites certain nerve fibers while reducing/minimizing excitation of certain other nerve fibers, e.g., such that peak 508 is increased and peak 704 is decreased. For example, a system may determine that leads 230 may be moved and/-or stimulation parameters settings 242 may be adjusted to increase peak 508 (e.g., increase excitation of the certain nerve fibers) while also decreasing peak 704 (e.g., reducing valley 704 or making peak 704 less negative, representing a decrease of the excitation of certain other nerve fibers).

As another example, improved and or optimal electrical stimulation therapy may be electrical stimulation that reduces/minimizes fiber excitation of some fibers while increasing excitation of other nerve fibers and muscle contraction, e.g., the EMG response of a muscle. For example, a system may determine that leads 230 may be moved and/or stimulation parameters settings 242 may be adjusted to increase peak 704 (e.g., increase valley 704 or make peak 704 more negative, representing an increase of the excitation of certain fibers of a nerve) while increasing peak 604 (e.g., increasing the response and corresponding EMG of a muscle) and decreasing peak 508 (e.g., decreasing excitation of other fibers of a nerve).

In some examples, the criteria for the evoked signal may include a number of peaks within the evoked signal exceeding a threshold. For example, composite signal 902 in FIG. 9 includes positive peaks 508 and 604. In some examples, processing circuitry may determine that two positive peaks satisfy the threshold number of peaks criteria.

In some examples, the criteria for evoked signal 902 may include a number of peaks within evoked signal 902 with amplitudes exceeding an amplitude threshold. The example of FIG. 9 includes amplitude threshold 912, and the amplitudes of both of peaks 508 and 604 exceed amplitude threshold 912. The processing circuitry may determine that the amplitude of two peaks 508 and 604 exceeding amplitude threshold 912 satisfies the criteria.

In some examples, the criteria for evoked signal 902 may include a time-based average of evoked signal 902 exceeding a threshold value within a latency window. In the example of FIG. 9, plot 900 includes time-based average 914. The processing circuitry may determine that time-based average 914 does not satisfy a time-based average threshold.

In some examples, the criteria for evoked signal 902 may include a composition criteria, where the type of evoked signal that composes evoked signal 902 may indicate a therapy response group in which to classify the patient. For example, a composition criteria may include a composite evoked signal that is composed primarily of EMG-like signals of muscle origin, indicating that a patient should be classified into a particular therapy response group.

In some examples, the criteria for an evoked signal may include any one or more criterion that satisfies a threshold (exceeds a threshold, is less than a threshold, or falls within a threshold range). In some examples, the criteria may include one or more of determining whether a number of signal peaks satisfies a threshold, whether an amplitude of one or more signal peaks satisfies a threshold, whether an area under one or more signal peaks satisfies a threshold, whether a signal peak width satisfies a threshold, whether a time between signal peaks satisfies a threshold, whether a ratio of signal peak amplitudes satisfies a threshold, whether a ratio of signal peak widths satisfies a threshold, whether a ratio of areas under signal peaks satisfies a threshold, whether a latency of a signal peak satisfies a threshold, whether a number of signal valleys satisfies a threshold, whether one or more signal valley amplitudes satisfies a threshold, whether an area above a signal valley satisfies a threshold, whether a signal valley width satisfies a threshold, whether a time between signal valleys satisfies a threshold, whether a ratio of signal valley amplitudes satisfies a threshold, whether a ratio of signal valley widths satisfies a threshold, whether a ratio of areas above signal valleys satisfies a threshold, whether a signal valley latency satisfies a threshold, whether a root-mean-square signal value satisfies a threshold, whether a signal skew satisfies a threshold, whether a signal kurtosis satisfies a threshold, whether a signal frequency satisfies a threshold, whether a signal spectral content satisfies a threshold, whether a Hjorth feature satisfies a threshold, whether a signal amplitude growth curve satisfies a threshold, whether a signal amplitude growth curve inflection point amplitude satisfies a threshold, whether a signal amplitude growth curve inflection point latency satisfies a threshold, whether a signal amplitude growth curve saturation point satisfies a threshold, whether a signal strength duration curve chronaxie satisfies a threshold, whether a signal strength duration curve rheobase satisfies a threshold, whether another signal strength duration curve feature satisfies a threshold, whether a signal maximum rate of change feature (e.g., maximum of the derivative of the signal) satisfies a threshold, whether a signal minimum rate of change feature (e.g., the minimum of the derivative of the signal) satisfies a threshold, or any other suitable signal feature satisfies a threshold.

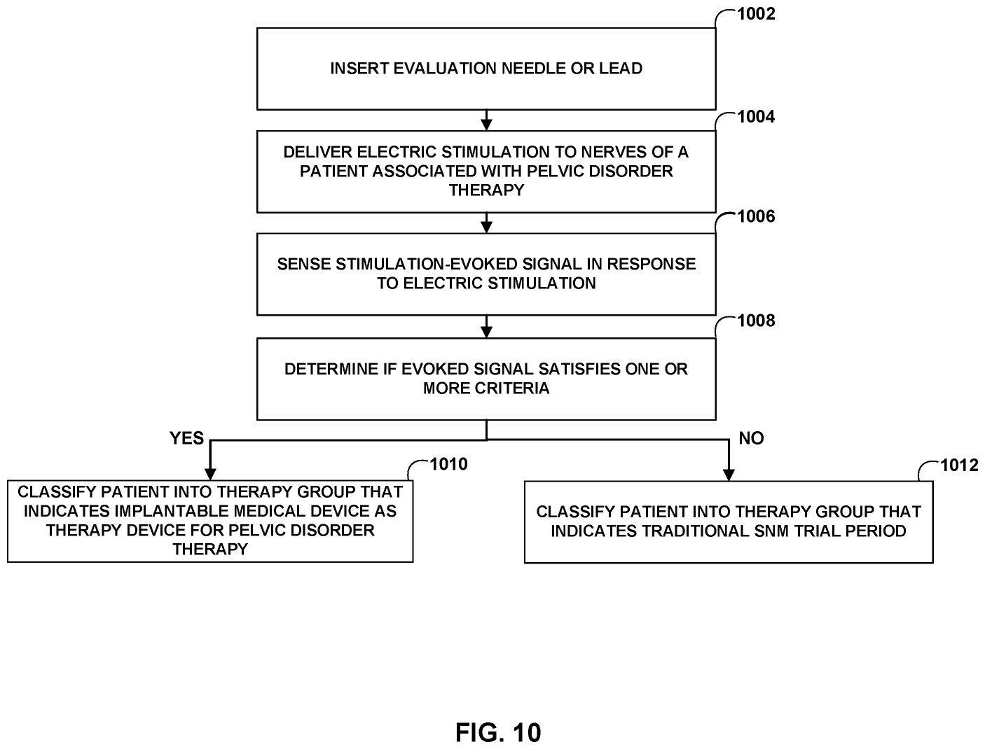

FIG. 10 is a flow diagram illustrating an example method of classifying a patient into a therapy response group indicating a therapy device type, in accordance with one or more techniques of this disclosure.

A physician may insert an evaluation needle or lead into patient tissue (1002). For example, the evaluation needle may be inserted through a foramen in the spine and positioned near the patient tissue for stimulation. In some examples, an introducer is inserted to allow the evaluation lead to be used. In some examples, the evaluation needle/lead may have a minimal profile to provide the least intrusive evaluation procedure to the patient.

Processing circuitry of a stimulation device attached to the evaluation lead or needle may deliver (e.g., via stimulation circuitry of the stimulation device) one or more electrical stimulation pulses to one or more nerves of the patient via one or more electrodes attached to or formed on the evaluation needle or lead prior to implantation of an implantable medical device, wherein the one or more nerves are associated with pelvic disorder therapy (1004). Block 1004 of FIG. 10 may be substantially similar to block 402 of FIGS. 4A and 4B.

Processing circuitry of the stimulation device may sense (e.g., via sensing circuitry of stimulation device), via the electrode, an evoked signal generated by one or more signal sources in response to delivery of the one or more electrical stimulation pulses (1006). (1006) of FIG. 10 may be substantially similar to (404) of FIGS. 4A and 4B.

The processing circuitry of the stimulation device may determine whether the one or more evoked signals satisfy one or more criteria (1008). Block 1008 of FIG. 10 may be substantially similar to block 414 of FIG. 4B. In order to classify patient 14 into a therapy response group substantially coincident with the delivery of the one or more electrical stimulation pulses, the processing circuitry may be configured to determine whether the evoked signal satisfies the one or more criteria.

In response to determining that the evoked signal satisfies the criteria, the processing circuitry may classify the patient into a therapy response group that indicates that an implantable IMD as the therapy device for pelvic disorder therapy (1010). For example, the processing circuitry may classify the patient into a therapy response group that indicates an implantable IMD would be effective for alleviating pelvic disorder symptoms of the patient.

In response to determining that the evoked signal does not satisfy the peak amplitude threshold, processing circuitry may classify the patient into a therapy response group that indicates that the patient should undergo an elongated evaluation period (1012). The processing circuitry may be unable to classify—substantially coincident with the delivery of the one or more stimulation pulses—the patient into a therapy response group that indicates whether or not an implantable IMD would be effective for alleviating pelvic disorder symptoms of the patient. The elongated evaluation period may include a traditional sacral neuromodulation trialing period.

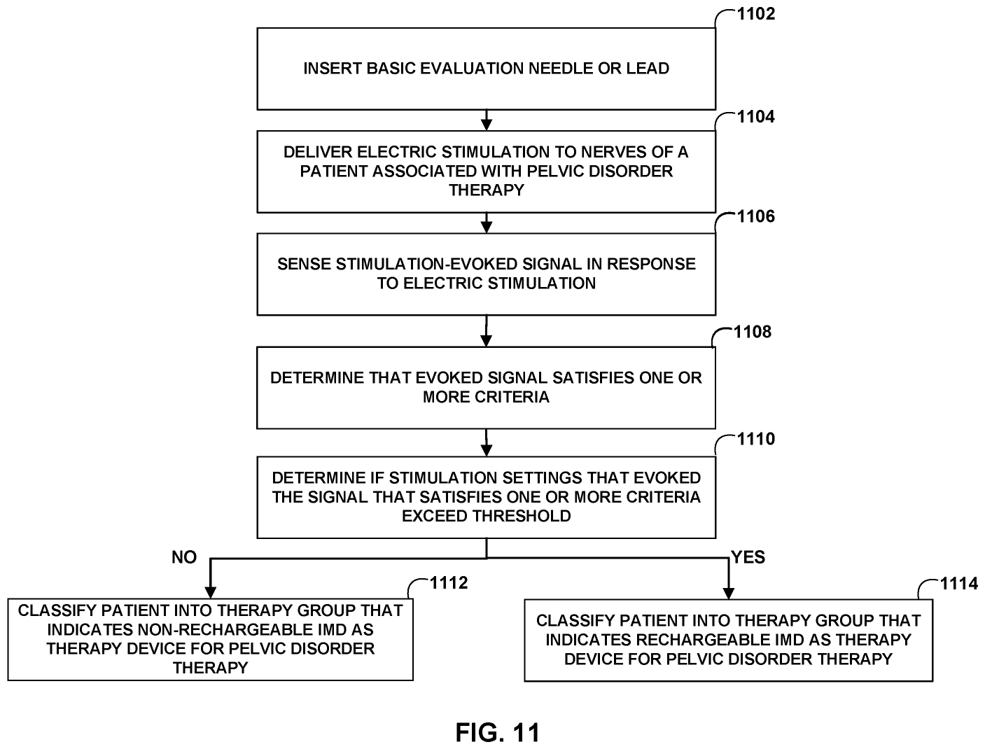

FIG. 11 is a flow diagram illustrating another example method of classifying a patient into a therapy response group indicating a therapy device type, in accordance with one or more techniques of this disclosure. A physician may insert an evaluation needle or lead into patient tissue (1102). Block 1102 of FIG. 11 may be substantially similar to block 1002 of FIG. 10.

Processing circuitry of a stimulation device attached to the evaluation lead or needle may deliver (e.g., via stimulation circuitry of the stimulation device) one or more electrical stimulation pulses to one or more nerves of the patient via one or more electrodes attached to or formed on the evaluation needle or lead prior to implantation of an implantable medical device, wherein the one or more nerves are associated with pelvic disorder therapy (1104). Block 1104 of FIG. 11 may be substantially similar to block 1004 of FIG. 10.

Processing circuitry of the stimulation device may sense (e.g., via sensing circuitry of stimulation device), via the electrode, an evoked signal generated by one or more signal sources in response to delivery of the one or more electrical stimulation pulses (1106). Block 1106 of FIG. 11 may be substantially similar to block 1006 of FIG. 10.

The processing circuitry of the stimulation device may determine that the one or more evoked signals satisfy one or more criteria (1108). In order to classify patient 14 into a therapy response group substantially coincident with the delivery of the one or more electrical stimulation pulses, the processing circuitry may be configured to determine whether the evoked signal satisfies the one or more criteria. The criteria may include a threshold number of peaks present in the signal, a peak amplitude threshold, a threshold number of peaks that exceed a threshold amplitude, a threshold area under the curve of the evoked signal data, etc.

The processing circuitry of the stimulation device may determine whether the stimulation settings that evoked the signal that satisfies the one or more criteria exceed a threshold (1110). A storage device of the stimulation device may store stimulation parameter settings. In addition, the storage device may store the sensed evoked signal data. In some examples, the stimulation device may process the evoked signal data and select or adjust stimulation parameter settings, including cycling, based on the evoked signal data. In some examples, the evoked signals may generally increase in amplitude as the stimulation amplitude is increased.

In response to determining that the stimulation settings that evoked the signal that satisfies the criteria do not exceed a threshold, the processing circuitry may classify the patient into a therapy response group that indicates a non-rechargeable IMD as a therapy device to provide pelvic disorder therapy to the patient (1112). By the stimulation settings not exceeding a threshold, the processing circuitry may determine that delivering electrical stimulation and sensing for evoked signals required an amount of energy that would not exceed a threshold amount of energy available in a non-rechargeable battery for an IMD.

In response to determining that the stimulation settings that evoked the signal that satisfies the criteria do exceed a threshold, the processing circuitry may classify the patient into a therapy response group that indicates a rechargeable, implantable IMD as a therapy device to provide pelvic disorder therapy to the patient (1114). By the stimulation settings exceeding a threshold, the processing circuitry may determine that delivering electrical stimulation and sensing for evoked signals requires an amount of energy that would exceed a threshold amount of energy available in a non-rechargeable battery for an IMD.

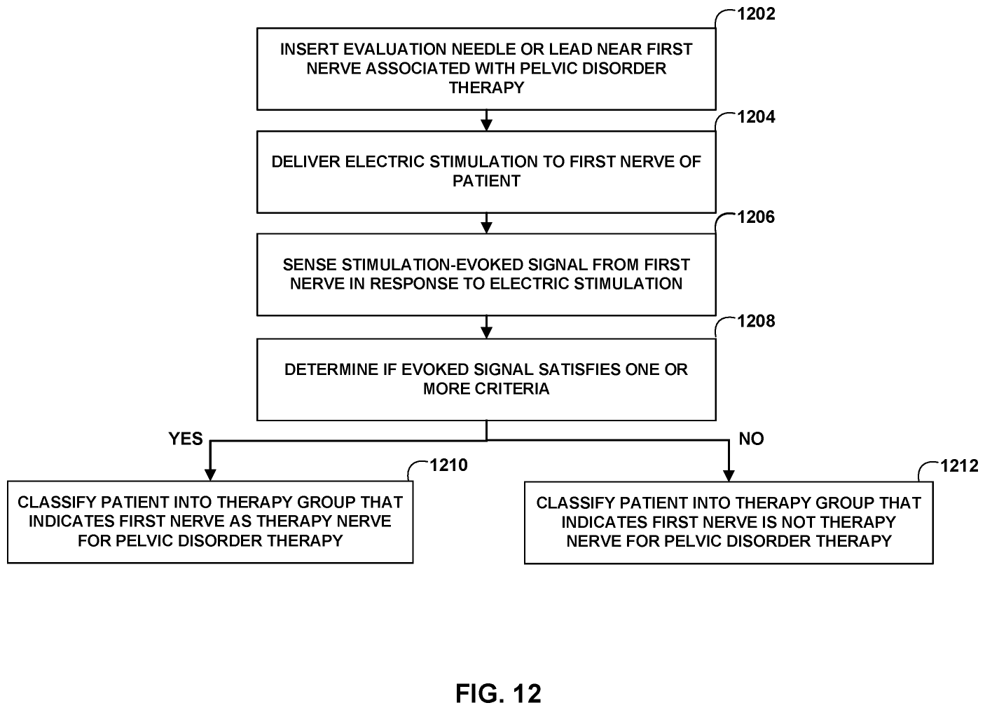

FIG. 12 is a flow diagram illustrating an example method of classifying a patient into a therapy response group indicating a nerve to use for pelvic disorder therapy, in accordance with one or more techniques of this disclosure. A physician may insert an evaluation needle or lead into patient tissue near a first nerve associated with pelvic disorder therapy (1202). Block 1202 of FIG. 12 may be substantially similar to block 1002 of FIG. 10.

Processing circuitry of a stimulation device attached to the evaluation lead or needle may deliver (e.g., via stimulation circuitry of the stimulation device) one or more electrical stimulation pulses to the first nerve of the patient via one or more electrodes attached to or formed on the evaluation needle or lead prior to implantation of an implantable medical device (1204). Block 1204 of FIG. 12 may be substantially similar to block 1004 of FIG. 10.

Processing circuitry of the stimulation device may sense (e.g., via sensing circuitry of stimulation device), via the electrode, an evoked signal generated by the first nerve in response to delivery of the one or more electrical stimulation pulses (1206). Block 1206 of FIG. 12 may be substantially similar to block 1006 of FIG. 10.

The processing circuitry of the stimulation device may determine whether the one or more evoked signals from the first nerve satisfy one or more criteria (1208). In order to classify patient 14 into a therapy response group substantially coincident with the delivery of the one or more electrical stimulation pulses, the processing circuitry may be configured to determine whether the evoked signal satisfies the one or more criteria. The criteria may include criteria to determine if the evoked signal is strong enough to sense based off of a given nerve at which the electrode is currently sensing. The criteria may include a threshold number of peaks present in the signal, a peak amplitude threshold, a threshold number of peaks that exceed a threshold amplitude, a threshold area under the curve of the evoked signal data, etc.

The processing circuitry of the stimulation device may determine that the evoked signal satisfies the one or more criteria, and classify the patient into a therapy response group that indicates the first nerve as the therapy nerve for pelvic disorder therapy (1210). By satisfying the criteria, the evoked signal may show that the current nerve undergoing electrical stimulation will provide effective pelvic disorder therapy for the patient for further stimulation.

The processing circuitry of the stimulation device may determine that the evoked signal does not satisfy the one or more criteria, and classify the patient into a therapy response group that indicates the first nerve is not the therapy nerve for pelvic disorder therapy (1212). By not satisfying the criteria, the evoked signal may show that the current nerve undergoing electrical stimulation will not provide effective pelvic disorder therapy for the patient for further stimulation. The processing circuitry may indicate or otherwise alert a physician using of the stimulation device that a location of the lead should change in order to provide better pelvic disorder therapy for the patient. The lead may then be moved to a second location to stimulate a different area of the first nerve, or to a second nerve.

The following numbered examples may illustrate one or more aspects of this disclosure:

Example 1: A system including: a device including processing circuitry configured to: deliver one or more electrical stimulation pulses to one or more nerves of a patient via one or more inserted electrodes on a lead or formed on an evaluation needle and prior to implantation of an implantable medical device, the one or more nerves being associated with stimulation therapy; sense an evoked signal generated by one or more signal sources in response to delivery of the one or more electrical stimulation pulses; determine, based at least in part on the evoked signal, a therapy response group of a plurality of therapy response groups for the patient, wherein the therapy response group indicates whether implantation of the implantable medical device is recommended; and output information indicative of the determined therapy response group.

Example 2: The system of example 1, wherein to determine the therapy response group for the patient, the processing circuitry is further configured to determine the therapy response group for the patient substantially coincident with the delivery of the one or more electrical stimulation pulses.

Example 3: The system of any of examples 1 and 2, wherein to determine the therapy response group for the patient, the processing circuitry is further configured to determine the therapy response group for the patient within a time period after delivery of the one or more electrical stimulation pulses, wherein the time period includes one of: less than one day; less than one hour; less than one minute; or less than one second.

Example 4: The system of any of examples 1-3, wherein the one or more nerves include one or more of sacral nerves and tibial nerves.

Example 5: The system of any of examples 1-4, wherein the processing circuitry is further configured to: determine that the evoked signal satisfies one or more criteria; and wherein to determine the therapy response group for the patient, the processing circuitry is further configured to classify, responsive to determining that the evoked signal satisfies the one or more criteria, the patient into the therapy response group that indicates that implantation of the implantable medical device is recommended for delivering stimulation therapy to the patient.

Example 6: The system of any of examples 1-4, wherein the processing circuitry is further configured to: determine that the evoked signal does not satisfy one or more criteria; and wherein to determine the therapy response group for the patient, the processing circuitry is further configured to classify, responsive to determining that the evoked signal does not satisfy the one or more criteria, the patient into the therapy response group that indicates that the patient is a candidate for longer term evaluation of whether to receive therapy with the implantable medical device.

Example 7: The system of example 6, wherein the longer term evaluation includes a multi-day evaluation of efficacy of the stimulation therapy.

Example 8: The system of any of examples 1-7, wherein the evoked signal includes a second evoked signal and the one or more electrical stimulation pulses includes a second set of one or more electrical stimulation pulses, and the processing circuitry is further configured to: determine that a first evoked signal, evoked prior to the second evoked signal from delivery of a first set of one or more electrical stimulation pulses delivered to a first location, does not satisfy one or more criteria; and wherein to deliver the second set of one or more electrical stimulation pulses the processing circuitry is further configured to deliver, responsive to the determination that the first evoked signal does not satisfy the one or more criteria, the second set of one or more electrical stimulation pulses at a second location different than the first location.

Example 9: The system of any of examples 1-8, wherein the stimulation therapy is pelvic disorder therapy, and wherein the processing circuitry is further configured to: determine that the evoked signal satisfies one or more criteria, wherein the criteria includes one or more of determining whether an amplitude of one or more peaks in the evoked signal exceeds a threshold, determining whether a number of peaks in the evoked signal exceeds a threshold, determining whether an area under the curve of the evoked signal exceeds a threshold, determining whether a peak is present in a latency window of the evoked signal; and wherein to determine the therapy response group for the patient, the processing circuitry is further configured to classify, based on the evoked signal satisfying the one or more criteria, the patient into the therapy response group that indicates that implantation of the implantable medical device is recommended for delivering stimulation therapy to the patient.

Example 10: The system of any of examples 1-9, wherein the processing circuitry is further configured to determine one or more criteria for the evoked signal based on a trained machine learning model.

Example 11: The system of example 10, wherein processing circuitry is further configured to train the machine learning model using evoked signal data from a plurality of patients undergoing known therapy programs.

Example 12: The system of any of examples 10 and 11, wherein the processing circuitry is further configured to periodically retrain the machine learning model using patient therapy program data collected over a period of time.

Example 13: The system of any of examples 1-12, wherein the processing circuitry is further configured to: determine that the evoked signal satisfies one or more criteria; and wherein to determine the therapy response group for the patient, the processing circuitry is further configured to classify, based on the evoked signal satisfying the one or more criteria, the patient into the therapy response group that indicates that the implantable medical device is a rechargeable implantable medical device.

Example 14: The system of any of examples 1-12, wherein the processing circuitry is further configured to: determine that the evoked signal satisfies one or more criteria; and wherein to determine the therapy response group for the patient, the processing circuitry is further configured to classify, based on the evoked signal satisfying the one or more criteria, the patient into the therapy response group that indicates that the implantable medical device is a non-rechargeable implantable medical device.

Example 15: The system of any of examples 1-14, wherein the evoked signal includes a second evoked signal and the one or more electrical stimulation pulses includes a second set of one or more electrical stimulation pulses, and the processing circuitry is further configured to: determine that a first evoked signal, evoked prior to the second evoked signal from delivery of a first set of one or more electrical stimulation pulses delivered with a first stimulation amplitude, does not satisfy one or more criteria; wherein to deliver the second set of one or more electrical stimulation pulses the processing circuitry is further configured to deliver, responsive to the determination that the first evoked signal does not satisfy the one or more criteria, the second set of one or more electrical stimulation pulses at a second stimulation amplitude different than the first stimulation amplitude; and output information indicative of the stimulation level at which the therapy response group was determined for the patient.

Example 16: The system of any of examples 1-15, wherein the one or more inserted electrodes are percutaneously implanted within the patient.

Example 17: The system of any of examples 1-16, wherein the evoked signal is one of a compound muscle action potential or a neural evoked compound action potential generated by patient muscle tissue or neural tissue, respectively.

Example 18: The system of any of examples 1-17, wherein the evoked signal is a composite stimulation-evoked signal including a composite of signals generated by one or more signal sources, wherein the signal sources include patient muscle tissue or patient neural tissue.

Example 19: A method including: delivering one or more electrical stimulation pulses to one or more nerves of a patient via one or more inserted electrodes on a lead or formed on an evaluation needle and prior to implantation of an implantable medical device, the one or more nerves being associated with stimulation therapy; sensing an evoked signal generated by one or more signal sources in response to delivery of the one or more electrical stimulation pulses; determining, based at least in part on the evoked signal, a therapy response group of a plurality of therapy response groups for the patient, wherein the therapy response group indicates whether implantation of the implantable medical device is recommended; outputting information indicative of the determined therapy response group.

Example 20: The method of example 19, wherein determining the therapy response group for the patient includes determining the therapy response group for the patient substantially coincident with the delivery of the one or more electrical stimulation pulses.

Example 21: The method of any of examples 19 and 20, wherein determining the therapy response group includes determining the therapy response group for the patient within a time period after delivery of the one or more electrical stimulation pulses, wherein the time period includes one of: less than one day; less than one hour; less than one minute; or less than one second.

Example 22: The method of any of examples 19-21, wherein the one or more nerves include one or more of sacral nerves and tibial nerves.

Example 23: The method of any of examples 19-22, further including: determining that the evoked signal satisfies one or more criteria; and wherein determining the therapy response group for the patient includes classifying, responsive to determining that the evoked signal satisfies the one or more criteria, the patient into the therapy response group that indicates that implantation of the implantable medical device is recommended for delivering stimulation therapy to the patient.

Example 24: The method of any of examples 19-23, further including: determining that the evoked signal does not satisfy one or more criteria; and wherein determining the therapy response group for the patient includes classifying, responsive to determining that the evoked signal does not satisfy the one or more criteria, the patient into the therapy response group that indicates the patient is a candidate for longer term evaluation of whether to receive therapy with the implantable medical device.

Example 25: The method of example 24, wherein the longer term evaluation includes a multi-day evaluation of efficacy of the stimulation therapy.

Example 26: The method of any of examples 19-25, wherein the evoked signal includes a second evoked signal and the one or more electrical stimulation pulses includes a second set of one or more electrical stimulation pulses, and the method further includes: determining that a first evoked signal, evoked prior to the second evoked signal from delivery of a first set of one or more electrical stimulation pulses delivered to a first location, does not satisfy one or more criteria; and wherein delivering the second set of one or more electrical stimulation pulses includes delivering, responsive to determining that the first evoked signal does not satisfy the one or more criteria, the second set of one or more electrical stimulation pulses at a second location different than the first location.

Example 27: The method of any of examples 1-26, further including: determining that the evoked signal satisfies one or more criteria, wherein the criteria includes one or more of determining whether an amplitude of one or more peaks in the evoked signal exceeds a threshold, determining whether a number of peaks in the evoked signal exceeds a threshold, determining whether an area under the curve of the evoked signal exceeds a threshold, determining whether a peak is present in a latency window of the evoked signal; and wherein determining the therapy response group for the patient includes classifying, based on the evoked signal satisfying the one or more criteria, the patient into the therapy response group that indicates that implantation of the implantable medical device is recommended for delivering stimulation therapy to the patient.

Example 28: The method of any of examples 19-27, further including determining the one or more criteria for the evoked signal based on a trained machine learning model.

Example 29: The method of example 28, further including training the machine learning model using evoked signal data from a plurality of patients undergoing known therapy programs.

Example 30: The method of any of examples 28 and 29, further including periodically retraining the machine learning model using patient therapy program data collected over a period of time.

Example 31: The method of any of examples 19-30, further including: determining that the evoked signal satisfies one or more criteria; and wherein determining the therapy response group for the patient includes classifying, based on the evoked signal satisfying the one or more criteria, the patient into the therapy response group that indicates that the implantable medical device is a rechargeable implantable medical device.

Example 32: The method of any of examples 19-30, further including: determining that the evoked signal satisfies one or more criteria; and wherein determining the therapy response group for the patient includes classifying, based on the evoked signal satisfying the one or more criteria, the patient into the therapy response group that indicates that the implantable medical device is a non-rechargeable implantable medical device.

Example 33: The method of any of examples 19-32, wherein the evoked signal includes a second evoked signal and the one or more electrical stimulation pulses includes a second set of one or more electrical stimulation pulses, and the method further includes: determining that a first evoked signal, evoked prior to the second evoked signal from delivery of a first set of one or more electrical stimulation pulses delivered with a first stimulation amplitude, does not satisfy one or more criteria; wherein delivering the second set of one or more electrical stimulation pulses includes delivering, responsive determining that the first evoked signal does not satisfy the one or more criteria, the second set of one or more electrical stimulation pulses at a second stimulation amplitude different than the first stimulation amplitude; and outputting information indicative of the stimulation level at which the therapy response group was determined for the patient.

Example 34: A computer readable medium including instructions that, when executed, cause one or more processors to: deliver one or more electrical stimulation pulses to one or more nerves of a patient via one or more inserted electrodes on a lead or formed on an evaluation needle and prior to implantation of an implantable medical device, the one or more nerves being associated with pelvic disorder therapy; sense an evoked signal generated by one or more signal sources in response to the delivery of the one or more electrical stimulation pulses; determine, based at least in part on the evoked signal, a therapy response group of a plurality of therapy response groups for the patient, wherein the therapy response group indicates whether implantation of the implantable medical device is recommended; and output information indicative of the determined therapy response group.

Example 35: The system of any of examples 1-18, wherein the processing circuitry is further configured to: determine that the evoked signal satisfies one or more criteria, wherein the criteria includes one or more of determining whether a number of signal peaks satisfies a threshold, whether an amplitude of one or more signal peaks satisfies a threshold, whether an area under one or more signal peaks satisfies a threshold, whether a signal peak width satisfies a threshold, whether a time between signal peaks satisfies a threshold, whether a ratio of signal peak amplitudes satisfies a threshold, whether a ratio of signal peak widths satisfies a threshold, whether a ratio of areas under signal peaks satisfies a threshold, whether a latency of a signal peak satisfies a threshold, whether a number of signal valleys satisfies a threshold, whether one or more signal valley amplitudes satisfies a threshold, whether an area above a signal valley satisfies a threshold, whether a signal valley width satisfies a threshold, whether a time between signal valleys satisfies a threshold, whether a ratio of signal valley amplitudes satisfies a threshold, whether a ratio of signal valley widths satisfies a threshold, whether a ratio of areas above signal valleys satisfies a threshold, whether a signal valley latency satisfies a threshold, whether a root-mean-square signal value satisfies a threshold, whether a signal skew satisfies a threshold, whether a signal kurtosis satisfies a threshold, whether a signal frequency satisfies a threshold, whether a signal spectral content satisfies a threshold, whether a Hjorth feature satisfies a threshold, whether a signal amplitude growth curve satisfies a threshold, whether a signal amplitude growth curve inflection point amplitude satisfies a threshold, whether a signal amplitude growth curve inflection point latency satisfies a threshold, whether a signal amplitude growth curve saturation point satisfies a threshold, whether a signal strength duration curve satisfies a threshold, whether a signal maximum rate of change satisfies a threshold, or whether a signal minimum rate of change satisfies a threshold; and wherein to determine the therapy response group for the patient, the processing circuitry is further configured to classify, based on the evoked signal satisfying the one or more criteria, the patient into the therapy response group that indicates that implantation of the implantable medical device is recommended for delivering pelvic disorder therapy to the patient.

Example 36: The system of any of examples 1-18 or 35, wherein the device includes a first device and the processing circuitry includes first processing circuitry, wherein the system further includes a second device including second processing circuitry, wherein the second processing circuitry is in communication with the first processing circuitry, and wherein the second processing circuitry is configured to receive signal data of the evoked signal from the first processing circuitry; and determine one or more criteria for the evoked signal based on a trained machine learning model.

Example 37: The system of example 36, wherein the second processing circuitry is further configured to train the machine learning model using evoked signal data from a plurality of patients undergoing known therapy programs.

Example 38: The system of example 36 or 37, wherein the second processing circuitry is further configured to periodically retrain the machine learning model using patient therapy program data collected over a period of time.

The techniques described in this disclosure may be implemented, at least in part, in hardware, software, firmware or any combination thereof. For example, various aspects of the described techniques may be implemented within processing circuitry, which may include one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also form one or more processors or processing circuitry configured to perform one or more of the techniques of this disclosure.

Such hardware, software, and firmware may be implemented, and various operation may be performed within the same device, within separate devices, and/or on a coordinated basis within, among or across several devices, to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components. Processing circuitry described in this disclosure, including a processor or multiple processors, may be implemented, in various examples, as fixed-function circuits, programmable circuits, or a combination thereof. Fixed-function circuits refer to circuits that provide particular functionality with preset operations. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive stimulation parameters or output stimulation parameters), but the types of operations that the fixed-function circuits perform are generally immutable. In some examples, one or more of the units may be distinct circuit blocks (fixed-function or programmable), and in some examples, one or more of the units may be integrated circuits.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

What is claimed is:

1. A system comprising:
a device comprising processing circuitry configured to:
deliver one or more electrical stimulation pulses to one or more nerves of a patient via one or more inserted electrodes on a lead or via a conductive portion on an evaluation needle and prior to implantation of an implantable medical device, the one or more nerves being associated with stimulation therapy;
sense an evoked signal generated by one or more signal sources in response to delivery of the one or more electrical stimulation pulses;
determine, based at least in part on the evoked signal, a therapy response group of a plurality of therapy response groups for the patient, wherein the therapy response group indicates whether implantation of the implantable medical device is recommended; and
output information indicative of the determined therapy response group.

2. The system of claim 1, wherein to determine the therapy response group for the patient, the processing circuitry is further configured to determine the therapy response group for the patient substantially coincident with the delivery of the one or more electrical stimulation pulses.

3. The system of claim 1, wherein to determine the therapy response group for the patient, the processing circuitry is further configured to determine the therapy response group for the patient within a time period after delivery of the one or more electrical stimulation pulses, wherein the time period includes one of:
less than one day;
less than one hour;
less than one minute; or
less than one second.

4. The system of claim 1, wherein the one or more nerves include one or more of sacral nerves and tibial nerves.

5. The system of claim 1, wherein the processing circuitry is further configured to:
determine that the evoked signal satisfies one or more criteria; and
wherein to determine the therapy response group for the patient, the processing circuitry is further configured to classify, responsive to determining that the evoked signal satisfies the one or more criteria, the patient into the therapy response group that indicates that implantation of the implantable medical device is recommended for delivering stimulation therapy to the patient.

6. The system of claim 1, wherein the processing circuitry is further configured to:
determine that the evoked signal does not satisfy one or more criteria; and
wherein to determine the therapy response group for the patient, the processing circuitry is further configured to classify, responsive to determining that the evoked signal does not satisfy the one or more criteria, the patient into the therapy response group that indicates that the patient is a candidate for longer term evaluation of whether to receive therapy with the implantable medical device.

7. The system of claim 1, wherein the evoked signal comprises a second evoked signal and the one or more electrical stimulation pulses comprises a second set of one or more electrical stimulation pulses, and the processing circuitry is further configured to:
determine that a first evoked signal, evoked prior to the second evoked signal from delivery of a first set of one or more electrical stimulation pulses delivered to a first location, does not satisfy one or more criteria; and
wherein to deliver the second set of one or more electrical stimulation pulses the processing circuitry is further configured to deliver, responsive to the determination that the first evoked signal does not satisfy the one or more criteria, the second set of one or more electrical stimulation pulses at a second location different than the first location.

8. The system of claim 1, wherein the processing circuitry is further configured to:
determine that the evoked signal satisfies one or more criteria, wherein the criteria includes one or more of determining whether a number of signal peaks satisfies a threshold, whether an amplitude of one or more signal peaks satisfies a threshold, whether an area under one or more signal peaks satisfies a threshold, whether a signal peak width satisfies a threshold, whether a time between signal peaks satisfies a threshold, whether a ratio of signal peak amplitudes satisfies a threshold, whether a ratio of signal peak widths satisfies a threshold, whether a ratio of areas under signal peaks satisfies a threshold, whether a latency of a signal peak satisfies a threshold, whether a number of signal valleys satisfies a threshold, whether one or more signal valley amplitudes satisfies a threshold, whether an area above a signal valley satisfies a threshold, whether a signal valley width satisfies a threshold, whether a time between signal valleys satisfies a threshold, whether a ratio of signal valley amplitudes satisfies a threshold, whether a ratio of signal valley widths satisfies a threshold, whether a ratio of areas above signal valleys satisfies a threshold, whether a signal valley latency satisfies a threshold, whether a root-mean-square signal value satisfies a threshold, whether a signal skew satisfies a threshold, whether a signal kurtosis satisfies a threshold, whether a signal frequency satisfies a threshold, whether a signal spectral content satisfies a threshold, whether a Hjorth feature satisfies a threshold, whether a signal amplitude growth curve satisfies a threshold, whether a signal amplitude growth curve inflection point amplitude satisfies a threshold, whether a signal amplitude growth curve inflection point latency satisfies a threshold, whether a signal amplitude growth curve saturation point satisfies a threshold, whether a signal strength duration curve satisfies a threshold, whether a signal maximum rate of change satisfies a threshold, or whether a signal minimum rate of change satisfies a threshold; and
wherein to determine the therapy response group for the patient, the processing circuitry is further configured to classify, based on the evoked signal satisfying the one or more criteria, the patient into the therapy response group that indicates that implantation of the implantable medical device is recommended for delivering pelvic disorder therapy to the patient.

9. The system of claim 1, wherein the processing circuitry is further configured to:
train a machine learning model using evoked signal data from a plurality of patients undergoing known therapy programs;
determine one or more criteria for the evoked signal based on the trained machine learning model; and
periodically retrain the machine learning model using patient therapy program data collected over a period of time.

10. The system of claim 1, wherein the processing circuitry is further configured to:
determine that the evoked signal satisfies one or more criteria; and
wherein to determine the therapy response group for the patient, the processing circuitry is further configured to classify, based on the evoked signal satisfying the one or more criteria, the patient into the therapy response group that indicates that the implantable medical device is a rechargeable implantable medical device.

11. The system of claim 1,
wherein the device comprises a first device and the processing circuitry comprises first processing circuitry,
wherein the system further comprises a second device comprising second processing circuitry,
wherein the second processing circuitry is in communication with the first processing circuitry, and
wherein the second processing circuitry is configured to
train a machine learning model using evoked signal data from a plurality of patients undergoing known therapy programs;
receive signal data of the evoked signal from the first processing circuitry;
determine one or more criteria for the evoked signal based on the trained machine learning model; and
periodically retrain the machine learning model using patient therapy program data collected over a period of time.

12. The system of claim 1, wherein the processing circuitry is further configured to:
determine that the evoked signal satisfies one or more criteria; and
wherein to determine the therapy response group for the patient, the processing circuitry is further configured to classify, based on the evoked signal satisfying the one or more criteria, the patient into the therapy response group that indicates that the implantable medical device is a non-rechargeable implantable medical device.

13. The system of claim 1, wherein the evoked signal comprises a second evoked signal and the one or more electrical stimulation pulses comprises a second set of one or more electrical stimulation pulses, and the processing circuitry is further configured to:
determine that a first evoked signal, evoked prior to the second evoked signal from delivery of a first set of one or more electrical stimulation pulses delivered with a first stimulation amplitude, does not satisfy one or more criteria;
wherein to deliver the second set of one or more electrical stimulation pulses the processing circuitry is further configured to deliver, responsive to the determination that the first evoked signal does not satisfy the one or more criteria, the second set of one or more electrical stimulation pulses at a second stimulation amplitude different than the first stimulation amplitude; and
output information indicative of the stimulation level at which the therapy response group was determined for the patient.

14. A method comprising:
delivering one or more electrical stimulation pulses to one or more nerves of a patient via one or more inserted electrodes on a lead or formed on an evaluation needle and prior to implantation of an implantable medical device, the one or more nerves being associated with stimulation therapy;
sensing an evoked signal generated by one or more signal sources in response to delivery of the one or more electrical stimulation pulses;
determining, based at least in part on the evoked signal, a therapy response group of a plurality of therapy response groups for the patient, wherein the therapy response group indicates whether implantation of the implantable medical device is recommended; and
outputting information indicative of the determined therapy response group.

15. The method of claim 14, wherein determining the therapy response group comprises determining the therapy response group for the patient within a time period after delivery of the one or more electrical stimulation pulses, wherein the time period includes one of:
less than one day;
less than one hour;
less than one minute; or
less than one second.

16. The method of claim 14, further comprising:
determining that the evoked signal satisfies one or more criteria; and
wherein determining the therapy response group for the patient comprises classifying, responsive to determining that the evoked signal satisfies the one or more criteria, the patient into the therapy response group that indicates that implantation of the implantable medical device is recommended for delivering stimulation therapy to the patient.

17. The method of claim 14, wherein the evoked signal comprises a second evoked signal and the one or more electrical stimulation pulses comprises a second set of one or more electrical stimulation pulses, and the method further comprises:
determining that a first evoked signal, evoked prior to the second evoked signal from delivery of a first set of one or more electrical stimulation pulses delivered to a first location, does not satisfy one or more criteria; and
wherein delivering the second set of one or more electrical stimulation pulses comprises delivering, responsive to determining that the first evoked signal does not satisfy the one or more criteria, the second set of one or more electrical stimulation pulses at a second location different than the first location.

18. The method of claim 14, further comprising:
determining that the evoked signal satisfies one or more criteria; and
wherein determining the therapy response group for the patient comprises classifying, based on the evoked signal satisfying the one or more criteria, the patient into the therapy response group that indicates that the implantable medical device is a rechargeable or non-rechargeable implantable medical device.

19. The method of claim 14, wherein the evoked signal comprises a second evoked signal and the one or more electrical stimulation pulses comprises a second set of one or more electrical stimulation pulses, and the method further comprises:
determining that a first evoked signal, evoked prior to the second evoked signal from delivery of a first set of one or more electrical stimulation pulses delivered with a first stimulation amplitude, does not satisfy one or more criteria;
wherein delivering the second set of one or more electrical stimulation pulses comprises delivering, responsive determining that the first evoked signal does not satisfy the one or more criteria, the second set of one or more electrical stimulation pulses at a second stimulation amplitude different than the first stimulation amplitude; and
outputting information indicative of the stimulation level at which the therapy response group was determined for the patient.

20. A computer readable medium comprising instructions that, when executed, cause one or more processors to:
deliver one or more electrical stimulation pulses to one or more nerves of a patient via one or more inserted electrodes on a lead or formed on an evaluation needle and prior to implantation of an implantable medical device, the one or more nerves being associated with stimulation therapy;
sense an evoked signal generated by one or more signal sources in response to the delivery of the one or more electrical stimulation pulses;
determine, based at least in part on the evoked signal, a therapy response group of a plurality of therapy response groups for the patient, wherein the therapy response group indicates whether implantation of the implantable medical device is recommended; and
output information indicative of the determined therapy response group.
